(19) United States
(12) Patent Application Publication
(10) Pub. No.: US 2005/0048551 A1
(43) Pub. Date: Mar. 3, 2005

(54) Analyte evaluating device, method for evaluating analyte and method for manufacturing analyte evaluating device

(75) Inventors: Kaoru Nakajima, Kawasaki (JP); Shozo Fujita, Kawasaki (JP); Tsuyoshi Fujihara, Kawasaki (JP); Shunsaku Takeishi, Kawasaki (JP)

Correspondence Address: ARMSTRONG, KRATZ, QUINTOS, HANSON & BROOKS, LLP, 1725 K STREET, NW, SUITE 1000, WASHINGTON, DC, 20006, US

(73) Assignee: FUJITSU LIMITED, Kawasaki (JP)

(21) Appl. No.: 10/900,432

(22) Filed: Jul. 28, 2004

(30) Foreign Application Priority Data
Aug. 28, 2003 (JP) 2003-305041

(51) Int. Cl. C12Q 1/68; G01N 33/53; C12M 1/34

(52) U.S. Cl. 435/6; 435/7.1; 435/287.2

(57) ABSTRACT

An analyte evaluating device is provided that comprises a carrier body that can be bound with an analyte having a fluorescence-labeled part that can emit fluorescence by light received when the distance between the fluorescence-labeled part and the carrier body is enlarged, wherein the distance between the fluorescence-labeled part and the carrier body can be varied by a responding part equipped on at least one of the analyte and the carrier body. It is possible to perform a high-sensitivity evaluation without introducing a fluorescence-labeled part or a radioactive material into an evaluation object. Evaluation is possible for a very small amount of sample. Furthermore, evaluation is possible even when multiple types of evaluation objects are present in a mixture. Miniaturized, complex, and integrated analyte evaluating devices can be provided.

### BACKGROUND OF THE INVENTION

1. Field of the Invention

The present invention relates to the technology for evaluating an evaluation object represented by a biochip and DNA chip.

2. Description of the Related Art

The human genome project that have advanced since the beginning of 1990's is a multinational effort in which each country takes a responsibility for part of the work to decode the whole human genetic codes, and it was announced in the summer of 2000 that the draft version of decoding was completed. It is expected that what kind of function each sequencing position for the decoded human genome sequencing information is related with, will be clarified as the functional genomic science and structural genomic science develop in future.

This human genome project has brought a great change in paradigm for scientific technologies and industries in relation with life science. For example, diabetes mellitus has been classified according to the condition of the disease that blood glucose level is elevated, and regarding the causes of the manifestation, classification has been made into type I (being unable to produce insulin in the body), type II (being unable to control the amount of insulin in the body), etc., based on how much the insulin productivity is in the body of a patient.

A human genome project presents us all of the information of amino acid sequencing structures of proteins such as enzymes and receptors in relation with detection, synthesis, decomposition, and other regulations of blood glucose and insulin, and the information of the DNA sequencing of genomes in relation with control of the amount of such proteins present.

Using such information should make it possible to classify diabetes mellitus as a phenomenon that the blood glucose level is not regulated in a normal manner, into subtypes, based on which of the respective proteins in relation with a group of processes such as detection, synthesis and decomposition of blood glucose and insulin, are in disorder, and accordingly, appropriate diagnosis and treatment should become possible.

In particular, genome-based drug discovery for developing a medicine for a specific protein based on the human genome sequencing has been promoted energetically. It is now expected that time will come when genome-based drugs are administered based on the understanding of the state of such a group of the proteins functionally related with each other in order to alleviate symptoms and to cure a disease.

However, the technology for simply and conveniently measuring the amounts of such a group of proteins that are functionally related with each other, is still in the developing stage as a proteome analysis technology. One measurement method using two-dimensional electrophoresis in combination with mass spectrometry has been established. However, this method requires relatively large-scale apparatuses, and therefore, development of new technologies is needed to clinically ascertain the conditions of a disease of a patient, for example, in a laboratory or at the bedside of a patient in a hospital.

With such a need at the background, studies called micro-Total Analysis System (μ-TAS) and Lab-on-a-chip have attracted interest. These technologies provide microscopic devices obtained by forming grooves of a micrometer size (microchannels) on a several-centimeter-square substrate of glass or silicone in order to perform chemical analyses or chemical reactions. Owing to the fact that liquid or gaseous samples are made to flow into microscopic flow channels (several hundred to several μm in width), advantages are given such as reduced amounts of the samples and wastes, high-speed processing, etc. Furthermore, there is a possibility to miniaturize even chemical plants. Thus, application of such technologies to biotechnology is being expected. It is to be noted that μ-TAS is translated into Japanese as “Shusekika Kagaku Bunseki System” (Accumulated Chemical Analysis System), “Maikuro Kagaku-Seikagaku Bunseki System” (Microchemical-Biochemical Analysis System), etc. It is a chemical analysis system with miniaturized sensors, analyzers, or the like, integrating, on a chip, functions of devices for use in analytical chemical laboratories.

Among these, the biochip technologies represented by DNA chips (or DNA microarrays) attract attention as effective means for gene analysis. Biochips comprises substrates made of glass, silicon, plastics, etc. on the surface of which numerous different test substances of biomacromolecules such as DNAs and proteins, are highly densely arrayed as spots. They can simplify examination of nucleic acids and proteins in the fields of clinical diagnosis and pharmacotherapy (for example, Japanese Unexamined Patent Application Publication No. 2001-235468 (paragraph numbers 0002-0009), and “Journal of American Chemical Society”, vol. 119, p. 8916-8920, 1997.

As test substances, DNAs and nucleotides are used, for example. Accordingly, in many cases biochips are called DNA chips.

When fragments of unknown DNAs or analytes are made to flow into such a DNA chip, targeted DNAs are captured by hybridization with the test substances, utilizing the property of DNAs that they are bound or combined with complementary DNAs. If a fluorescence-labeled part is attached to the unknown DNAs beforehand, the captured analytes are detected by the fluorescence signals from respective spots on the DNA chip. Thus, the state of from several thousand to tens of thousands of DNAs or RNAs of analytes can be observed at once by analyzing the data on a computer.

In such a so-called DNA chip, fluorescent pigments are introduced during the amplification (multiplication) of DNAs or the targeted objects that has been performed previously by PCR (polymerase chain reaction), so that the amounts of DNAs in a specimen bound with complementary DNA strands or chains located in a array are measured quantitatively by the intensities of fluorescence.

However, amplification of proteins corresponding to the PCR is not possible. Furthermore, there is a problem that uniform introduction of fluorescence-labeled parts is not possible owing to the difference in reactivity between each protein and the pigment, if numerous types of proteins are present in a specimen as a mixture.

Furthermore, the biochips such as the above had the following problems. Firstly, for specific biomacromolecules, it was necessary to prepare spots of numerous different test substances in a highly dense array, and accordingly, it was necessary to dispose reaction vessels for each targeted biomacromolecule. Furthermore, each vessel has a size that is visible to the naked eye. This poses an obstacle against the miniaturization.

Secondly, while formation of a microscopic flow path pattern on a substrate utilizing photolithography, followed by formation of grooves by etching or the like, and then formation of the flow paths by putting a cover on, is a process commonly adopted when manufacturing complex biochips such as μ-TASs, there are no means to install DNA test substances once a chip is sealed. Thus, the work of forming spots of test substances in a highly dense array must be performed during the manufacture of biochips. This poses a problem that the subsequent biochip manufacturing processing steps would influence on DNAs.

### SUMMARY OF THE INVENTION

Accordingly, it is an object of the present invention to solve the above-described problems and provide a new technology for evaluating evaluation objects such as proteins with a high sensitivity. Other objects and advantages of the present invention will become evident from the following explanations.

According to one aspect of the present invention, an analyte evaluating device is provided which comprises a carrier body that can be bound with an analyte having a fluorescence-labeled part that can emit fluorescence by light received when the distance between the fluorescence-labeled part and the carrier body is enlarged, the distance between the fluorescence-labeled part and the carrier body being variable by an external action, a light irradiation device for the fluorescence-labeled part to emit light, and a fluorescence detecting device for detecting the fluorescence emitted by the fluorescence-labeled part.

By the present invention, an analyte evaluating device for evaluating evaluation objects such as proteins with a high sensitivity is realized. It is possible to perform the evaluation without introducing fluorescence-labeled parts or radioactive materials into the evaluation objects. Evaluation for a tiny amount of sample is possible. It is also possible to perform the evaluation, even if there are various kinds of evaluation objects in a mixed state in a sample. Furthermore, miniaturized, complex, and integrated analyte evaluating devices are possible.

Preferable are that the distance between the fluorescence-labeled part and the carrier body can be varied by a responding part that is located on at least one of the analyte and the carrier body; that the external action is an electromagnetic or chemical action; particularly that the carrier body is an electrode and the electromagnetic action is realized by applying an electric potential difference between the electrode and a counter electrode; that the carrier body can be chemically bound with the analyte; that the carrier body has a Au layer on the surface; that the carrier body has an analyte binding part having at least one type of group selected from the class consisting of a carboxy group, thiol group, amino group, thioisocyanate group, isocyanate group and α-keto halide group; that the analyte binding part is bound with the Au layer via a thiol group; that the carrier body can be bound with an analyte by one of the following reactions A to E,
A. a reaction between a carboxy group and an amino group via an imidazole-bound intermediate that is activated by 1-(3-dimethylamino-propyl)-3-ethyl-carbodiimide hydrochloride,
B. a reaction between a carboxy group and an amino group via an N-hydroxysuccinimide-bound or an N-hydroxysuccinimide sulfonic acid-bound intermediate that is activated by 1-(3-dimethylamino-propyl)-3-ethyl-carbodiimide hydrochloride,
C. a reaction between a thiol group and a maleimide group,
D. a reaction between an isocyanate group and an amino group, and
E. a reaction between an α-keto halide group, and an amino group or thiol group;
   that the analyte has an evaluation object binding part that has a property to specifically bind to at least one evaluation object selected from the group consisting of proteins, DNAs, RNAs, antibodies, natural or artificial single-stranded nucleotides, natural or artificial double-stranded nucleotides, aptamers, products obtained by limited decomposition of antibodies with a protease, organic compounds having affinity to proteins, biomacromolecules having affinity to proteins, complex materials thereof, and arbitrary combinations thereof; particularly that the evaluation object is a protein; that the responding part can be charged positively or negatively; that the responding part comprises at least one material selected from the group consisting of proteins, DNAs, RNAs, antibodies, natural or artificial single-stranded nucleotides, natural or artificial double-stranded nucleotides, aptamers, products obtained by limited decomposition of antibodies with a protease, organic compounds having affinity to proteins, biomacromolecules having affinity to proteins, complex materials thereof, and arbitrary combinations thereof; particularly that the responding part comprises a natural or artificial single-stranded nucleotide, or a natural or artificial double-stranded nucleotide; that the responding part comprises a Fab fragment or (Fab)₂ fragment of an antibody; that the responding part comprises a fragment derived from an IgG antibody, or a fragment derived from a Fab fragment or (Fab)₂ fragment of an IgG antibody; that the responding part comprises a nucleotide aptamer; that the light irradiation device uses one or more optical fibers; that the light irradiation device is a laser light irradiation device; that the fluorescence-labeled part can be excited by evanescent waves; that a lens is installed between the light irradiation device and the carrier body; that the lens is a confocal lens; that light can be irradiated from a direction in parallel with the surface of the carrier body; that the carrier body is bound with the analyte; that a plurality of the same type or different types of carrier bodies are installed; that a plurality of the same type or different types of analytes are installed; that a plurality of carrier bodies are installed, and an electric potential is applied to each one of plural carrier bodies that is different from those for the other carrier bodies so that each carrier body can be bound with a different type of analyte; and that electric potentials are applied to a plurality of carrier body installation sites that are different from those of the other carrier body installation sites so that a different type of carrier body is formed on each installation site.

According to another aspect of the present invention, an analyte evaluating device is provided that has a flow path, an evaluation object capturing part for capturing an evaluation object with a first capture body, and a capture body capturing part for capturing a first capture body that has not captured an evaluation object with a second capture body, installed in this order.

By the present invention, separation of evaluation objects is made easier.

Preferable are that the analyte is evaluated by measuring the radiation amount of the analyte or the fluorescence intensity by at least one of the emission or extinction of fluorescence; that the main body of an analyte evaluating device (analyte evaluating device's main body) having an evaluation object capturing part and a capture body capturing part installed in this order, comprises a carrier body that can be bound with an analyte having a fluorescence-labeled part that can emit fluorescence by light received when the distance between the fluorescence-labeled part and the carrier body is enlarged, the distance between the fluorescence-labeled part and the carrier body being variable by an external action, a light irradiation device for the fluorescence-labeled part to emit light, and a fluorescence detecting device for detecting the fluorescence emitted by the fluorescence-labeled part.

An analyte evaluating device according to the first aspect comprising a flow path, an evaluation object capturing part for capturing an evaluation object with a first capture body, and a capture body capturing part for capturing a first capture body that has not captured an evaluation object with a second capture body, installed in this order, is also a preferable aspect.

In any case, preferable are that the evaluation object capturing part comprises a carrier body that can be bound with or detached from the first capture body by the presence or absence of an external action; that the capture body capturing part comprises a carrier body that can be bound with or detached from the second capture body by the presence or absence of an external action; that the presence or absence of the external action is the presence or absence of an electromagnetic or chemical action; that the presence or absence of the electromagnetic action is created by applying or not applying an electric potential difference between an electrode and a counter electrode; that the first capture body has a property to be specifically bound with an evaluation object; that the first capture body can be specifically bound with the evaluation object and the second capture body at the same site; that at least one of the first and second capture bodies is bound to a Au layer via a thiol group; that the first capture body has a property to be specifically bound with at least one evaluation object selected from the group consisting of proteins, DNAs, RNAs, antibodies, natural or artificial single-stranded nucleotides, natural or artificial double-stranded nucleotides, aptamers, products obtained by limited decomposition of antibodies with a protease, organic compounds having affinity to proteins, biomacromolecules having affinity to proteins, complex materials thereof, and arbitrary combinations thereof; particularly that the evaluation object is a protein; that at least one of the first and second capture bodies can be charged positively or negatively; that at least one of the first and second capture bodies comprises at least one material selected from the group consisting of proteins, DNAs, RNAs, antibodies, natural or artificial single-stranded nucleotides, natural or artificial double-stranded nucleotides, aptamers, products obtained by limited decomposition of antibodies with a protease, organic compounds having affinity to proteins, biomacromolecules having affinity to proteins, complex materials thereof, and arbitrary combinations thereof; particularly that at least one of the first and second capture bodies comprises a natural or artificial single-stranded nucleotide, or a natural or artificial double-stranded nucleotide; that at least one of the first and second capture bodies comprises a Fab fragment or (Fab)₂ fragment of an antibody; that at least one of the first and second capture bodies comprises a fragment derived from an IgG antibody, or a fragment derived from a Fab fragment or (Fab)₂ fragment of an IgG antibody; that at least one of the first and second capture bodies comprises a nucleotide aptamer; that at least one of the first and second capture bodies comprises at least one type of group selected from the class consisting of a carboxy group, thiol group, amino group, thioisocyanate group, isocyanate group and α-keto halide group; that the first capture body can be bound with an evaluation object by one of the above-described reactions A to E, or the second capture body can be bound with the first capture body, or both of the binding is possible; that the carrier body of the evaluation object capturing part has a Au layer on the surface, and the first capture body can be bound to and detached from the Au layer via a thiol group; that the carrier body of the capture body capturing part has a Au layer on the surface, and the second capture body can be bound to and detached from the Au layer via a thiol group; and that at least one of the outlets of the evaluation object capturing part and the capture body capturing part has a bottle-neck part to prevent the first or second capture body from exiting from the outlet.

According to still another aspect of the present invention, provided is a method for evaluating an analyte comprising: using the above-described analyte evaluating device; binding the analyte with the carrier body; changing the distance between the fluorescence-labeled part and the carrier body by an external action; irradiating light from the light irradiation device; and detecting fluorescence emitted from the fluorescence-labeled part with the fluorescence detecting device.

By the present invention, an analyte evaluating method with a high sensitivity is realized for evaluation objects such as proteins. It is also possible to perform the evaluation without introducing fluorescence-labeled parts or radioactive materials into the evaluation objects. It is also possible to perform the evaluation, even if the amount of the analyte is small. It is also possible to perform the evaluation, even if there are various kinds of evaluation objects in a mixed state in a sample.

Preferable are that light is irradiated from a direction in parallel with the surface of the carrier body; that the analyte is bound with an evaluation object before the analyte is bound with the carrier body; that each carrier body is given an electric potential different from those of the other carrier bodies so that a different type of analyte is disposed on each carrier body; that an electrode is used as the carrier body, and the electromagnetic action is realized by providing a potential difference having either one of a constant value, a pulse value, a value changing in a stepwise manner, a periodically changing value and a combination thereof, between the electrode and a counter electrode; and that at least one physical property selected from the group consisting of generation or non-generation of fluorescence emission, the rate of increase in the fluorescence intensity, the rate of decrease in the fluorescence intensity, the peak fluorescence intensity and the rate of change of the peak fluorescence intensity, is measured.

According to yet other aspects according to the present invention, provided are a method for manufacturing the above-described analyte evaluating device wherein the carrier body of the analyte evaluating device or of the main body thereof is prepared by treating a Au layer in an aqueous solution by either one of the above-described reactions A to E; a method for manufacturing the above-described analyte evaluating device wherein the Au layer of at least one of the first capture body and second capture body is treated in an aqueous solution with either one of the above-described reactions A to E; a method for manufacturing the above-described analyte evaluating device wherein an electric potential is applied to each one of plural carrier bodies that is different from those for the other carrier bodies so that each carrier body is bound with a different type of analyte; a method for manufacturing the above-described analyte evaluating device wherein an electric potential is applied to each one of carrier body installation sites that is different from those for the other carrier body installation sites so that each one of plural carrier body installation sites is bound with a different type of carrier body; a method for manufacturing the above-described analyte evaluating device wherein a different type of analyte is given to each carrier body after a cover is installed onto the analyte evaluating device; and a method for manufacturing the above-described analyte evaluating device wherein a different type of carrier body is given to each carrier body installation site after a cover is installed onto the analyte evaluating device.

By the present invention, a device for evaluating an analyte for evaluation objects such as proteins with a high sensitivity can be manufactured. Furthermore, miniaturized, complex, and integrated analyte evaluating devices are possible.

By the present invention, a device for evaluating an analyte for evaluation objects such as proteins with a high sensitivity, a method for evaluating an analyte therewith, and a manufacturing method therefor are provided.

By the present invention, it is possible to perform the evaluation without introducing fluorescence-labeled parts or radioactive materials into the evaluation objects. It is also possible to perform the evaluation, even if the amount of the analyte is small. It is also possible to perform the evaluation, even if there are various kinds of evaluation objects in a mixed state in a sample. Furthermore, miniaturized, complex, and integrated analyte evaluating devices, a method for evaluating an analyte therewith, and a manufacturing method therefor can be provided.

It is to be noted that the “analyte” in the present invention refers to an object to be detected and evaluated for finally evaluating an evaluation object with an analyte evaluating device. A case in which an evaluation object itself is an analyte as well as a case in which an analyte bound with an evaluation object is detected and evaluated is included in the category of the present invention. An “analyte evaluating device” has a function to comprehend an evaluation object by detection and evaluation of an analyte, and is a concept corresponding to a biochip or a DNA chip.

However, a case in which an analyte is either included or not included as explained later is also included in the category of the analyte evaluating device according to the present invention. Those devices in which a plurality of analyte evaluating devices are arranged, for example, in a dense array, are also included in the category of the analyte evaluating device according to the present invention. Furthermore, those devices integrated with other devices having other functions are also included in the category of the analyte evaluating device according to the present invention.

In a narrow sense, the analyte evaluating device realized by the present invention can be utilized for detecting biomacromolecules that now attract keen attention. Furthermore, by utilizing an optimum device structure according to the present invention, not only the detection of biomacromolecules but also comprehending the electric characteristics, diffusion characteristics, etc. of biomacromolecules and artificial nano-structures is possible, so that application to medical fields can be considered. It is expected that the scope of the application will be expanded as the functions of biological materials obtained through the human genome project are elucidated in future.

### BRIEF DESCRIPTION OF THE DRAWINGS

FIG. 1 is a model view illustrating states of analytes bound with a carrier body being expanded or shrunk, emitting or extinguishing fluorescence;

FIG. 2 is a view illustrating an example of a molecular structure for installing an analyte binding part;

FIG. 3 is a diagram illustrating a reaction route to make a carrier body surface having a SAM thereon;

FIG. 4 is another diagram illustrating a reaction route to make a carrier body surface having a SAM thereon;

FIG. 5 is another diagram illustrating a reaction route to make a carrier body surface having a SAM thereon;

FIG. 6 is another diagram illustrating a reaction route to make a carrier body surface having a SAM thereon;

FIG. 7 is another diagram illustrating a reaction route to make a carrier body surface having a SAM thereon;

FIG. 8 is another diagram illustrating a reaction route to make a carrier body surface having a SAM thereon;

FIG. 9 is a model view illustrating an analyte evaluating device according to the present invention, wherein fluorescence generated by light irradiation onto analytes on the way of leaving the carrier body is being detected;

FIG. 10 is another model view illustrating an analyte evaluating device according to the present invention, wherein fluorescence generated by light irradiation onto analytes on the way of leaving the carrier body is being detected;

FIG. 11 is a model view illustrating single-stranded DNAs with a fluorescent pigment are hybridized with single-stranded DNAs without a fluorescent pigment and bound with the carboxy groups of a SAM so as to form an analyte;

FIG. 12 is a diagram showing the change of fluorescence intensity with time when a pulse voltage is changed in a stepwise manner, in a case in which a fluorescent pigment and biotin (evaluation object binding part) are introduced onto single-stranded DNAs, and avidin or a protein (evaluation object) is not bound with the biotin;

FIG. 13 is a diagram showing the change of fluorescence intensity with time when a pulse voltage is changed in a stepwise manner, in a case in which a fluorescent pigment and biotin (evaluation object binding part) are introduced onto single-stranded DNAs, and avidin (evaluation object) is bound with the biotin;

FIG. 14 a view illustrating an example of arrangement of an analyte evaluating device according to the present invention;

FIG. 15 is another view illustrating an example of arrangement of an analyte evaluating device according to the present invention;

FIG. 16 is another view illustrating an example of arrangement of an analyte evaluating device according to the present invention;

FIG. 17 is another view illustrating an example of arrangement of an analyte evaluating device according to the present invention;

FIG. 18 is a diagram showing that the changing amounts of fluorescence intensity are different between a case in which avidin is present and a case in which avidin is absent;

FIG. 19 is an enlarged model view illustrating a sensor array part that is a collection of a plurality of carrier bodies before introducing analytes;

FIG. 20 is an enlarged model view illustrating a sensor array part that is a collection of a plurality of carrier bodies during introducing analytes onto a carrier body;

FIG. 21 is an enlarged model view illustrating a sensor array part that is a collection of a plurality of carrier bodies after having introduced analytes onto a carrier body;

FIG. 22 is a model plan view illustrating a common biochip;

FIG. 23 is a model side view of the biochip in FIG. 22;

FIG. 24A is a model view illustrating an evaluation object capturing part according to the present invention;

FIG. 24B is another model view illustrating an evaluation object capturing part according to the present invention;

FIG. 24C is another model view illustrating an evaluation object capturing part according to the present invention;

FIG. 24D is another model view illustrating an evaluation object capturing part according to the present invention;

FIG. 24E is a model view illustrating a capture body capturing part according to the present invention;

FIG. 24F is another model view illustrating a capture body capturing part according to the present invention;

FIG. 24G is a model view illustrating an analyte evaluating device's main body according to the present invention;

FIG. 24H is another model view illustrating an analyte evaluating device's main body according to the present invention;

FIG. 24I is another model view illustrating an analyte evaluating device's main body according to the present invention;

FIG. 25 is a model view illustrating an analyte evaluating device having an evaluation object capturing part and a capture body capturing part in this order;

FIG. 26 is another model view illustrating an analyte evaluating device having an evaluation object capturing part and a capture body capturing part in this order;

FIG. 27A is a model view illustrating a state of an analyte being bound with a carrier body;

FIG. 27B is a model view illustrating a state of an analyte being detached from a carrier body;

FIG. 28A is a model view illustrating a state of an analyte being bound with a carrier body and shrunk;

FIG. 28B is a model view illustrating a state of an analyte being expanded from a carrier body;

FIG. 29 is a model view illustrating binding of a SAM with DNAs;

FIG. 30 is a model view illustrating binding of a SAM with DNAs as well as hybridization of DNAs;

FIG. 31 is a model view illustrating states of analytes bound with a carrier body being expanded or shrunk, emitting or extinguishing fluorescence;

FIG. 32 is a model view illustrating a SAM;

FIG. 33 is a diagram illustrating fluorescence intensities when the concentration of an evaluation object is varied;

FIG. 34 is another diagram illustrating fluorescence intensities when the concentration of an evaluation object is varied;

FIG. 35 is another diagram illustrating fluorescence intensities when the concentration of an evaluation object is varied;

FIG. 36 is a diagram illustrating the relationship between the fluorescence intensity and the concentration of a protein;

FIG. 37 is a model view illustrating a state of single-stranded DNAs (analytes) having a fluorescent pigment being in the vicinity of a SAM of a carrier body;

FIG. 38 is a model view illustrating a state of the amino groups of single-stranded DNAs (analytes) having a fluorescent pigment being bound with carboxy groups of a SAM to form amide bonds;

FIG. 39 is a model view illustrating a state of single-stranded DNAs without a fluorescent pigment being in the vicinity of a SAM of a carrier body; and

FIG. 40 is a model view illustrating a state of the amino groups of single-stranded DNAs without a fluorescent pigment being bound with carboxy groups of a SAM to form amide bonds;

### DESCRIPTION OF THE PREFERRED EMBODIMENTS

For the purpose of solving the above-described problems, in Japanese patent applications No. 2002-297934, 2002-297941, etc., disclosed are technologies in which proteins are specifically determined quantitatively without applying labeling reactions such as fluorescence labeling as well as element technologies applicable to array chip technologies through which information useful from the viewpoint of proteome for comprehending proteins as a group is obtained.

It is an object according to the present invention to improve the sensitivity in the evaluation in such technologies and to make it easier for such technologies to be applied as element technologies. It is to be noted that the evaluation according to the present invention means detection of the presence and absence of an evaluation object as well as quantitative measurement.

An analyte evaluating device according to the present invention comprises a carrier body that can be bound with an analyte having a fluorescence-labeled part that can emit fluorescence by light received when the distance between the fluorescence-labeled part and the carrier body is enlarged, the distance between the fluorescence-labeled part and the carrier body being variable by an external action, a light irradiation device for the fluorescence-labeled part to emit light, and a fluorescence detecting device for detecting the fluorescence emitted by the fluorescence-labeled part.

In this device, an evaluation object can be evaluated by binding the analyte to the carrier body, realizing a state of the fluorescence-labeled part being extinguished by the quenching effect, and then, making the fluorescence-labeled part to emit light by enlarging the distance between the fluorescence-labeled part and the carrier body by an external action in order to observe the behavior of increasing and decreasing of the emitted light.

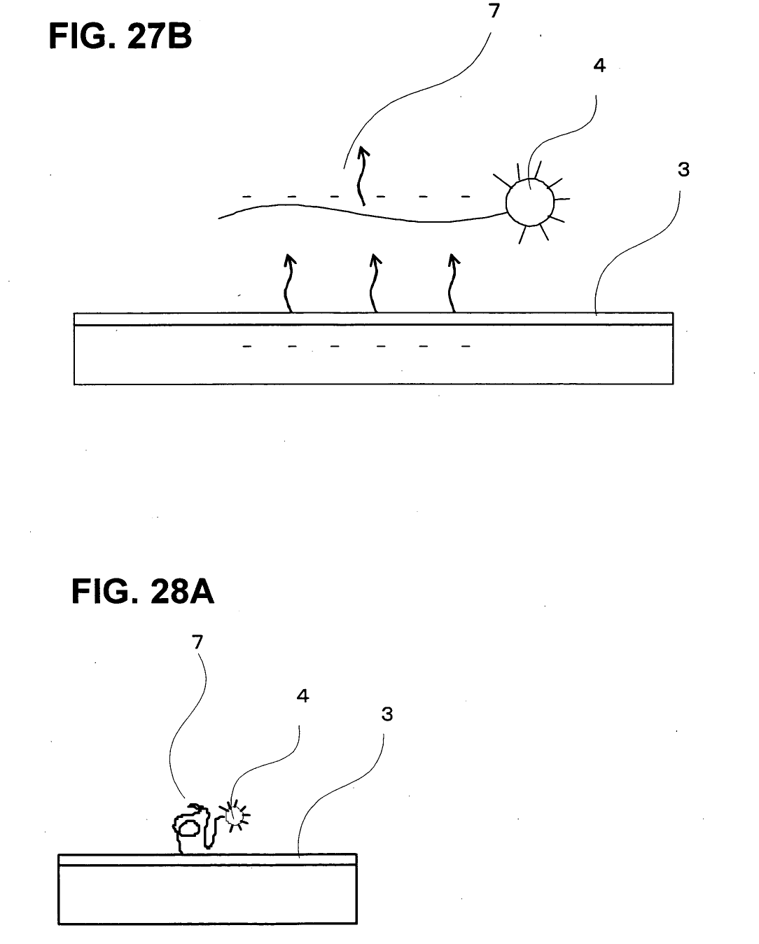

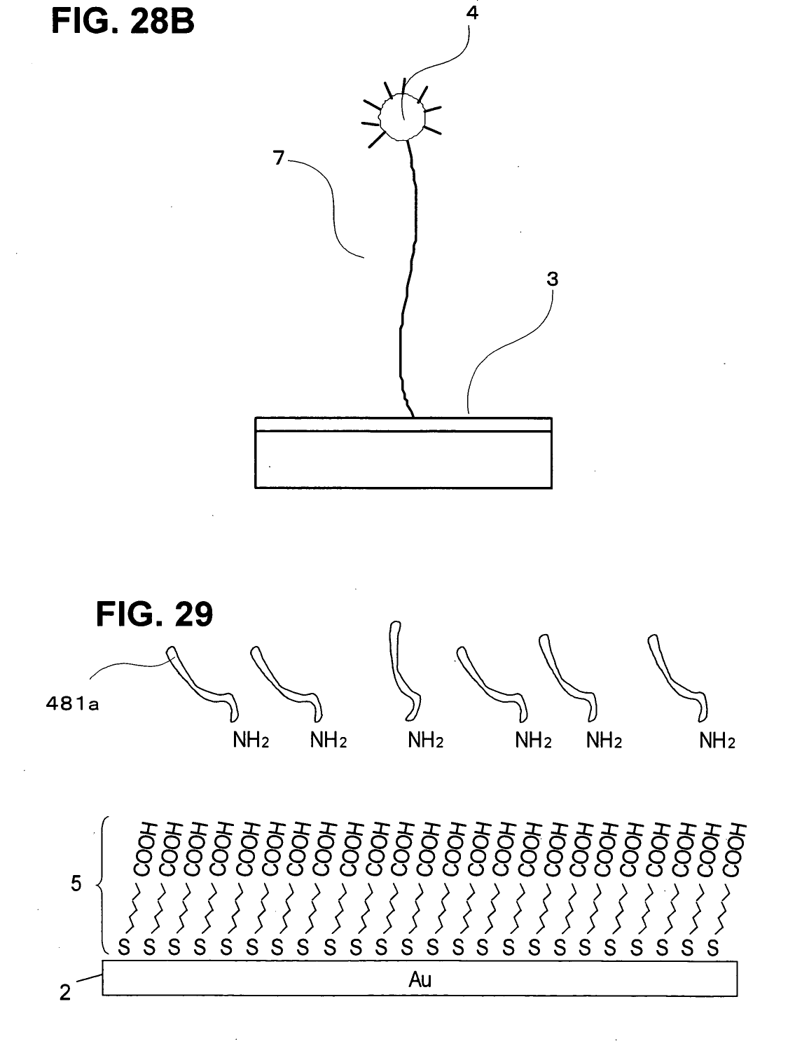

It is preferable that the distance between the fluorescence-labeled part and the carrier body can be varied by a responding part that has a function of detaching an analyte from the carrier body and/or expanding/shrinking an analyte, in response to an external action. Whether the responding part is located on the analyte or on the carrier body, the purpose can be achieved. It is to be noted that enlarging the distance between the fluorescence-labeled part and the carrier body can be realized by the detachment of the analyte from the carrier body or by expanding the responding part. FIG. 27A is a model view illustrating a state of an analyte 7 being bound with a carrier body 3 by adsorption, and FIG. 27B is a model view illustrating a state of an analyte 7 being detached from a carrier body 3. Furthermore, FIG. 28A is a model view illustrating a state of an analyte 7 being bound with a carrier body 3 and shrunk, and FIG. 28B is a model view illustrating a state of an analyte 7 being expanded from a carrier body 3. Regarding the case in which an analyte is detached from a carrier body, it is possible to consider that the responding part is located either on the analyte or on the carrier body or on both of them.

An evaluation object may be an analyte itself. It may also be a material that can be bound with an analyte or a material bound with an analyte as will be explained later. An evaluation object is preferably selected from the group consisting of proteins, DNAs, RNAs, antibodies, natural or artificial single-stranded nucleotides, natural or artificial double-stranded nucleotides, aptamers, products obtained by limited decomposition of antibodies with a protease, organic compounds having affinity to proteins, biomacromolecules having affinity to proteins, complex materials thereof, and arbitrary combinations thereof. Examples of the complex materials in the present invention include combined materials from DNAs and negatively-charged polymers, and combined materials from the above-described materials and other materials. An evaluation object is preferably a protein.

Hereupon, the “nucleotide” according to the present invention is any one selected from the group consisting of oligonucleotides and polynucleotides, or a mixture thereof. Such materials are often negatively charged. Single-stranded nucleotides and double-stranded nucleotides can be used. They can be specifically bound with analytes through hybridization. Proteins, DNAs and nucleotides can be used as a mixture. The biomacromolecules include those derived from living organisms, those processed from materials derived from living organisms, and synthesized molecules.

Hereupon, the above-described “products” are those obtained by limited decomposition of antibodies with a protease, and can comprise anything, as long as they conform to the gist of the present invention, including Fab fragments or (Fab)₂ fragments of antibodies, fragments derived from Fab fragments or (Fab)₂ fragments of antibodies, derivatives thereof, etc.

As an antibody, monoclonal immunoglobulin IgG antibodies can be used, for example. Fab fragments or (Fab)₂ fragments of IgG antibodies can be used as fragments derived from IgG antibodies, for example. Furthermore, fragments derived from those Fab fragments or (Fab)₂ fragments can be used. Examples of applicable organic compounds having affinity to proteins are enzyme substrate analogs such as nicotinamide adenine dinucleotide (NAD), enzyme activity inhibitors, neurotransmission inhibitors (antagonist), etc. Examples of biomacromolecules having affinity to proteins are proteins that can act as a substrate or a catalyst for proteins, element proteins constituting molecular composites, etc.

Any action that can vary the distance between a carrier body and a fluorescence-labeled part may be used as an external action. Electromagnetic or chemical actions are practical, and accordingly preferable.

For example, an electromagnetic action can be realized by using an electrode as a carrier body, installing a counter electrode, and giving a potential difference between these electrodes. The electromagnetic action can be realized by providing a potential difference having either one of a constant value, a pulse value, a value changing in a stepwise manner, a periodically changing value and a combination thereof, between the carrier and the counter electrode.

By employing such various types of potential difference, it is possible to evaluate the behaviors of expansion/shrinking, detachment from a carrier body and diffusion of an analyte under various conditions. It is also possible to separate those that are relatively hard to detach from a carrier body from those that are relatively easily detached in order to perform evaluation.

Any chemical actions can be used, including breaking chemical bonds such as covalent bonds and coordinate bonds that are existing, as well as preventing or furnishing ionic, hydrophobic, or polar interactions.

In response to the above-described various evaluation conditions, it is useful, for evaluating analytes, to measure at least one physical property selected from the group consisting of generation or non-generation of fluorescence emission, the rate of increase in the fluorescence intensity, the rate of decrease in the fluorescence intensity, the peak fluorescence intensity and the rate of change of the peak fluorescence intensity.

By these evaluations, the presence or absence of analyte binding and/or kinds of bound analytes and/or the amounts of bound analytes can be detected. Furthermore, the presence or absence of binding of biomacromolecules with the evaluation object binding part and/or kinds of bound biomacromolecules and/or the amounts of bound biomacromolecules can be detected.

A fluorescence-labeled part that emits or extinguishes fluorescence may be added by a covalent bond to an evaluation object as its part. It may also be added by a covalent bond to an analyte as its part before binding with an evaluation object, or may be included in a nucleotide or the like as shown in an example in which it is inserted (by intercalation) between adjacent complementary bonds, or integrated by substitution as a part of a nucleotide or the like. A fluorescence-labeled part is preferably located near the tip of an analyte.

A fluorescence-labeled part is selected from materials that are excited by the action of light and emit fluorescence. Examples suitable as a fluorescence-labeled part according to the present invention are indocarbocyanine 3 (trademark Cy3), etc.

Any material can be used as an analyte as long as it can be bound with a carrier body, and does not contradict the gist of the present invention. Preferable are those having, before binding with evaluation objects, a fluorescence-labeled part that can emit fluorescence by receiving light when the distance between the fluorescence-labeled part and a carrier body is enlarged.

It is preferable that the analyte has an evaluation object binding part having a property of specifically binding with an evaluation object. Evaluation is made possible by making evaluation objects such as proteins to bind with analytes via this evaluation object binding part, without applying fluorescence-labeling reactions or the like.

Such an evaluation object binding part preferably has a property of specifically bound to the above-described evaluation objects. There is no particular limitation to the type and the site of binding. However, it would be better to avoid binding with a particularly weak binding force.

A responding part has a function of being able to vary the distance between the fluorescence-labeled part and the carrier body by an external action. Varying the distance between the fluorescence-labeled part and the carrier body can be caused, as described before, by expansion/shrinking of the responding part as well as by detaching an analyte from the carrier body. For the purpose of varying the distance between the fluorescence-labeled part and the carrier body by an electromagnetic action, the responding part is preferably positively or negatively charged.

Such a responding part preferably comprises at least one material selected from the group consisting of proteins, DNAs, RNAs, antibodies, natural or artificial single-stranded nucleotides, natural or artificial double-stranded nucleotides, aptamers, products obtained by limited decomposition of antibodies with a protease, organic compounds having affinity to proteins, biomacromolecules having affinity to proteins, complex materials thereof, and arbitrary combinations thereof, because, in many occasions, it is easy to perform expansion and shrinking as well as detachment from the carrier body, and to be specifically bound with an evaluation object by acting also as an evaluation object binding part. Examples of a responding part charged positively or negatively include positively charged DNAs (guanidine DNAs) by utilizing guanidide bonding in the main chain, and negatively charged natural nucleotides.

Hereupon, the above-described “product” is obtained by limited decomposition of antibodies with a protease, and as long as the gist of the present invention is met, anything including Fab fragments or (Fab)₂ fragments of antibodies, fragments derived from those Fab fragments or (Fab)₂ fragments of antibodies, derivatives thereof, etc. can be included.

As an antibody, a monoclonal immunoglobulin IgG antibody can be used for example. Fab fragments or (Fab)₂ fragments of IgG antibodies can also be used as fragments derived from IgG antibodies, for example. Furthermore, fragments derived from those Fab fragments or (Fab)₂ fragments can also be used. Examples of applicable organic compounds having affinity to proteins are enzyme substrate analogs such as nicotinamide adenine dinucleotide (NAD), enzyme activity inhibitors, neurotransmission inhibitors (antagonist), etc. Examples of biomacromolecules having affinity to proteins are proteins that can act as a substrate or a catalyst for proteins, element proteins constituting molecular composites, etc.

As a responding part, natural nucleotides and artificial nucleotides can be used. Artificial nucleotides include completely artificial nucleotides and those derived from natural nucleotides. In some cases, use of artificial nucleotides may be advantageous in raising the sensitivity and improving the consistency of detection.

A responding part may also be a single-stranded nucleotide or a double-stranded nucleotide that is a pair of complementarily-related single-stranded nucleotides. In many cases, single-stranded nucleotides are preferable owing to the ease of expansion and shrinking. It is possible to use a different nucleotide for each electrode. Nucleotides with one or more residual groups are acceptable. That is, mononucleotides are acceptable.

Monoclonal antibodies and products obtained by limited decomposition of antibodies with a protease can also be used for a responding part. They are useful, since bonds created by the reactions similar to antigen-antibody reactions can be utilized, and they can act also as evaluation object binding parts.

For the responding part, it is also preferable to use monoclonal antibodies, Fab fragments or (Fab)₂ fragments of monoclonal antibodies, or fragments derived from Fab fragments or (Fab)₂ fragments of monoclonal antibodies. It is to be noted that the fragments derived from Fab fragments or (Fab)₂ fragments of monoclonal antibodies mean fragments obtained by fragmenting Fab fragments or (Fab)₂ fragments of monoclonal antibodies, and derivatives thereof.

Furthermore, it is more preferable to use, as a responding part, IgG antibodies, Fab fragments or (Fab)₂ fragments of IgG antibodies, or fragments derived from IgG antibodies, or Fab fragments or (Fab)₂ fragments of IgG antibodies. It is to be noted that the fragments derived from Fab fragments or (Fab)₂ fragments of IgG antibodies mean fragments obtained by fragmenting Fab fragments or (Fab)₂ fragments of IgG antibodies, and derivatives thereof. Nucleotide aptamers are also preferable. The reason is that those with a smaller molecular weight provide better detection sensitivity in general.

It is to be noted that not only cases in which the fluorescence-labeled part, evaluation object binding part, responding part and analyte are clearly distinct from each other but also cases in which part or the whole of one or more of them is also part or the whole of another or others, are included in the present invention. When the responding part comprises at least one material selected from the group consisting of proteins, DNAs, RNAs, antibodies, natural or artificial single-stranded nucleotides, natural or artificial double-stranded nucleotides, aptamers, products obtained by limited decomposition of antibodies with a protease, organic compounds having affinity to proteins, biomacromolecules having affinity to proteins, complex materials thereof, and arbitrary combinations thereof, it also has a part that functions as an evaluation object binding part in many cases.

Any material can be used as a carrier body according to the present invention, and there is no particular limitation to its shape, as long as it can be bound with an analyte, can vary the distance from a fluorescence-labeled part by an external action, and does not contradict the gist of the present invention. In this case, any type of binding can be utilized as long as it does not contradict the gist of the present invention, including biological binding, electrostatic binding, physical adsorption, chemical adsorption, etc., as well as chemical bonding such as covalent bonding and coordinate bonding.

For example, glasses, ceramics, plastics, metals, etc. can be carrier bodies according to the present invention. Those having a structure part (analyte binding part) that can be bound with an analyte on the surface, can also be used. The carrier body may be single-layered, or multi-layered. It may also has a structure other than layers.

Any material can be arbitrarily chosen for the carrier body depending on the purpose, but Au is particularly preferable. When a biomacromolecule is used as an analyte, it is easy to fix it onto the carrier body.

When an electromagnetic action is used as the external action, it is reasonable to use the whole or part of the carrier body as an electrode. An electroconductive material itself can be used as a carrier body. It is also possible to install a layer of an electroconductive material on the surface of a glass, ceramic, plastic, metal or the like. As such an electroconductive material, any material can be used including simple metal substances, alloys, laminates thereof, etc. Noble metals of which Au is representative, are preferably used owing to their chemical stability.

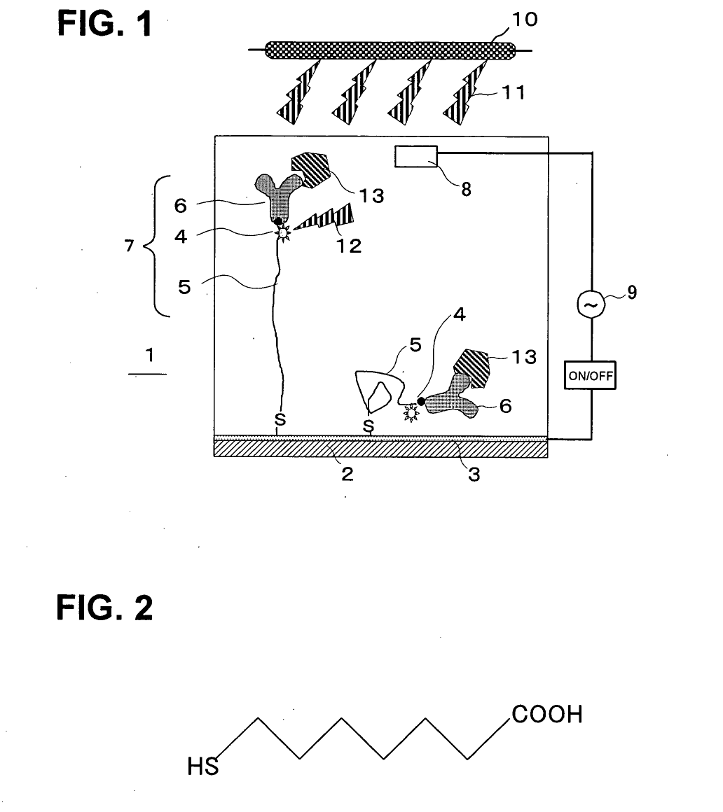

When the carrier can be bound with an analyte without specifically forming an analyte binding part, it is not necessary to install an analyte binding part on the surface. Taking a case in which an analyte comprises a nucleotide, and can be bound with a Au layer directly via its thiol group for example, there is an analyte evaluating device 1 as shown in FIG. 1 wherein analytes 7 are bound with a Au electrode (carrier body 3) installed on a sapphire substrate 2, the analytes having a fluorescence-labeled part 4, a responding part 5 having a natural single-stranded oligonucleotide structure, and an evaluation object binding part 6, and the binding being established by the reaction with the polished Au electrode at room temperature for 24 hours. “S” which is located in the lower portion of the single-stranded oligonucleotide structure represents that the analyte 7 is directly bound with the Au electrode 3 via a thiol group. In FIG. 1, a Fab fragment of a monoclonal immunoglobulin IgG is fixed at the tip of the oligonucleotide chain, as the evaluation object binding part 6 having a property to be specifically bound with an evaluation object.

On the left of FIG. 1, a state of an analyte being expanded, is illustrated. On the right, a state of an analyte being shrunk, is illustrated. The analyte 7 in the shrunk state can be expanded by applying a specific potential difference between the Au electrode 3 and a counter electrode 8 through an external electric field applying device 9. In this example, the distance between the fluorescence-labeled part and the carrier body varies not by detaching the analyte from the carrier body, but by the expansion of the responding part that is part of the analyte.

In this state, fluorescence 12 is provided as light 11 is irradiated from a light irradiation device 10. In FIG. 1, an evaluation object 13 is bound with the evaluation object binding part 6. When an analyte itself is an evaluation object, emission or extinction of fluorescence is evaluated without binding the analyte to an evaluation object, as shown in FIG. 31. This case does not require an evaluation object binding part.

In FIG. 1, the fluorescence-labeled part was introduced onto a single-stranded oligonucleotide beforehand. The thiol group and the fluorescence-labeled part may be introduced onto the end of the single strand or on the 5′ end of the strand or chain. In this example, the oligonucleotide strand was fixed onto the circular Au electrode having a diameter of 1 mm.

When an analyte binding part is installed as part of a carrier body, any material can be used as the analyte binding part, as long as it can be bound with an analyte. Examples are molecules that can be bound with an analyte via chemical bonding or intermolecular force. If the analyte binding part can expand/shrink or can detach an analyte, it can also act as a responding part. In this case, detaching from an analyte is not necessarily at a position where the analyte binding part is bound with the analyte. For example, detaching at a thiol group may be possible. Accordingly, it goes without saying that a case in which the position of detaching of an analyte from a carrier body may be different from the position where the carrier body is bound with the analyte, also belongs to the scope of the present invention in general.

Although it is generally ideal that the binding between a carrier body and an analyte is quantitative, there can be binding with a significantly large dissociation constant. If the dissociation constant is too large, the amount of bond may gradually decrease, for example, during washing with a buffer solution. From this viewpoint, it is generally preferable that the dissociation constant in the bisnding between a carrier body and an analyte be not mot than 10⁻⁵.

When an analyte binding part is installed, it may, for example, be a molecule with a structure having a thiol group on one of the ends and a carboxy group on the other end as shown in FIG. 2, wherein the thiol group is bound with the surface of Au electrode. It is to be noted that the thiol and carboxy groups are not necessarily located at the end of a molecule. Furthermore, any known metal other than Au can also be used for the surface of the electrode to be bound with a thiol group.

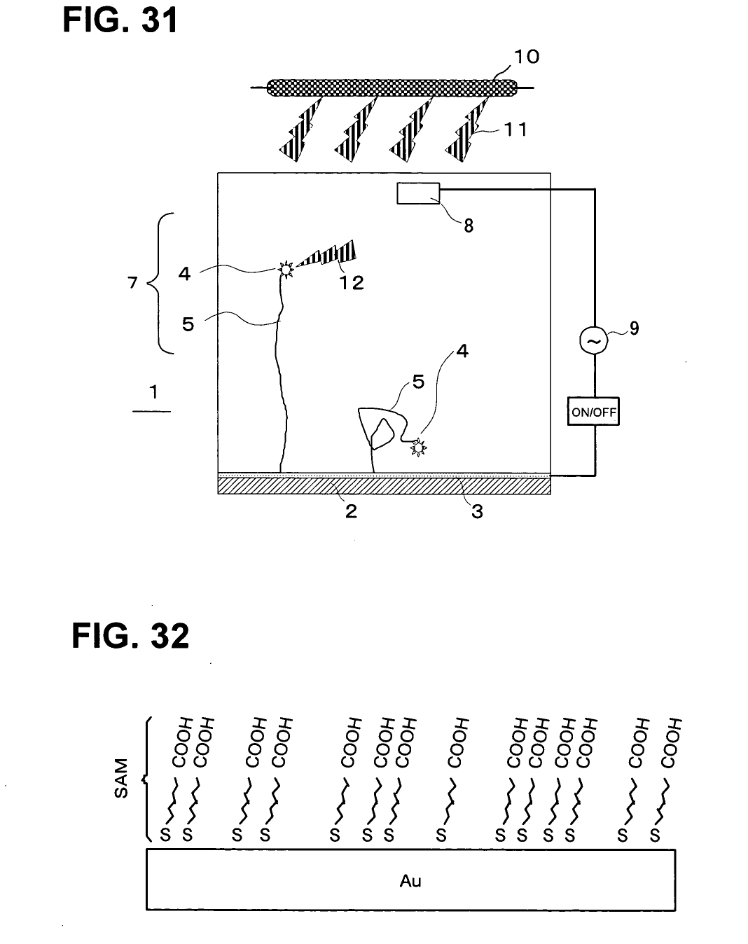

Such analyte binding parts are sometimes regarded as a membrane consisting of a single layer of molecules, and called a SAM (Self-Assembled Monolayer). That is to say, the carrier body according to the present invention may have a SAM on the surface as analyte binding parts. FIG. 32 is a model view for such a case. Hereupon, it is to be noted that a zigzag line connecting S and a carboxy group in FIGS. 2 and 32 means a bonding group. Any bonding group can be used as long as it does not contradict the gist of the present invention.

In the above description, the carboxy group is a group to be bound with an analyte. When an analyte is a polynucleotide having an amino group, for example, the carboxy group can be bound with the amino group through reactions A and B explained below. These reactions can be accomplished by sequentially treating with reagents below, the surface of a carrier body having a SAM on the surface.

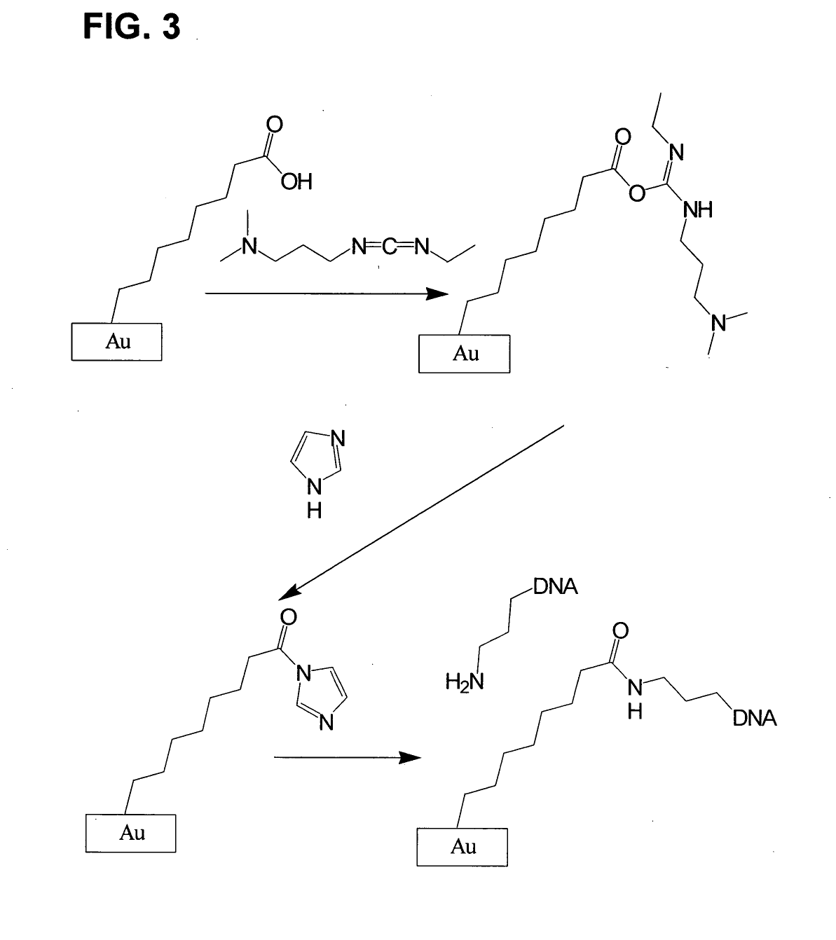

A. A reaction between a carboxy group and an amino group activated by 1-(3-dimethylamino-propyl)-3-ethyl-carbodiimide hydrochloride (may be called EDC, hereafter) and with an imidazole bound material as an intermediate. Specifically, amide bonding is finally formed via a route, for example, as shown in FIG. 3. In FIG. 3, a DNA with an amino group is bound.

B. A reaction between a carboxy group and an amino group activated by EDC and with an N-hydroxysuccinimide-bound or N-hydroxysuccinimide sulfonic acid-bound material as an intermediate.

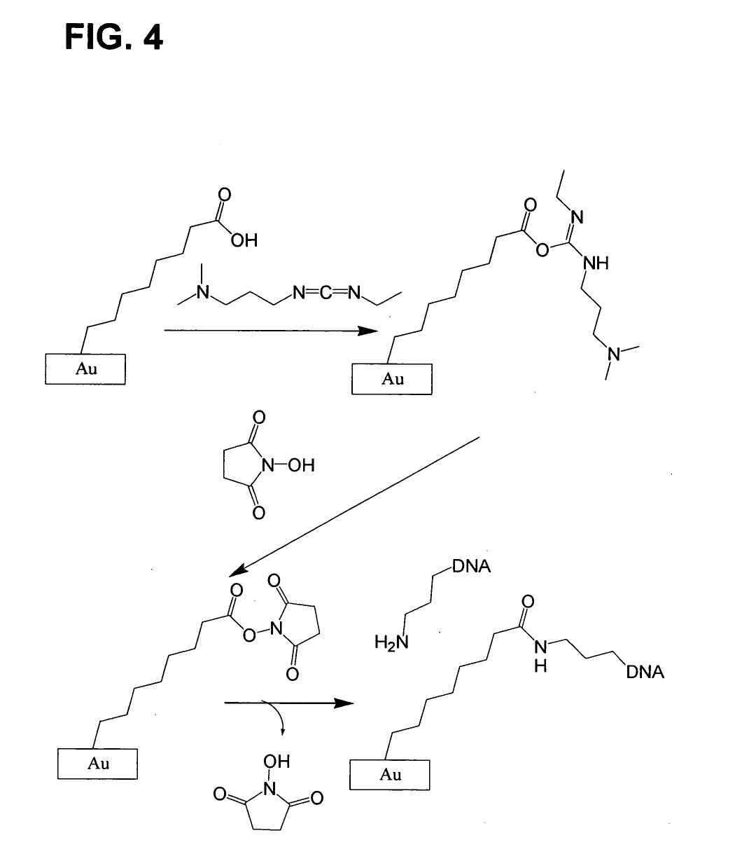

Amide bonding with a DNA having an amino group is finally formed via a route, for example, as shown in FIG. 4. Imidazole was present in the actual procedure. This was because increase in yield was expected owing to the fact the imidazole forms an intermediate just like N-hydroxysuccinimide or N-hydroxysuccinimide sulfonic acid.

The following C, D and E are examples of cases in which groups other than carboxy group are used for the carrier body, and/or groups other than amino group are used for the analyte. It goes without saying that any known method can be applied instead of the A, B, C, D and E. These chemical reactions may be facilitated by electric attachment caused by a controlled voltage application.

C. A reaction of a thiol group with a maleimide group.

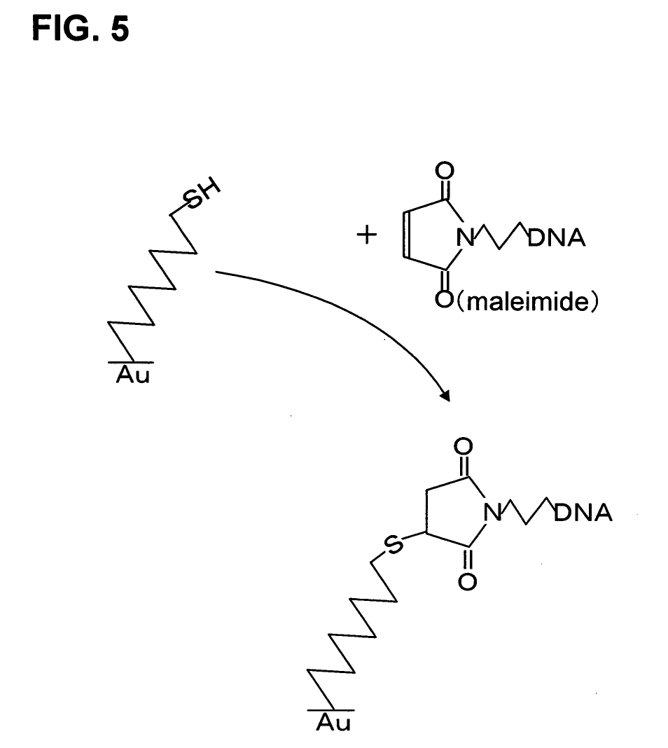

For example, thioether bonding is formed with a DNA having a maleimide group according to the route shown in FIG. 5.

D. A reaction of an isocyanate group with an amino group.

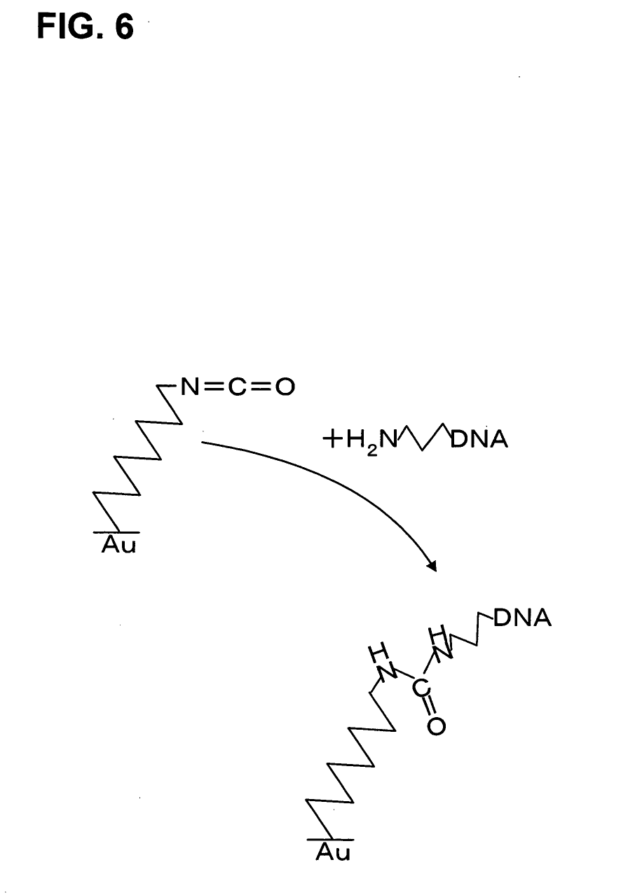

For example, urea bonding is formed with a DNA having an amino group according to the route shown in FIG. 6.

E. A reaction of an α-keto halide group with an amino group or thiol group.

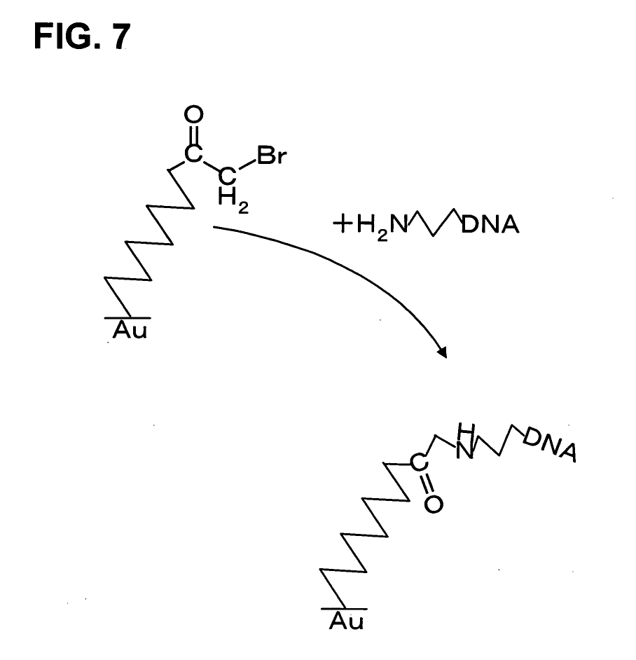

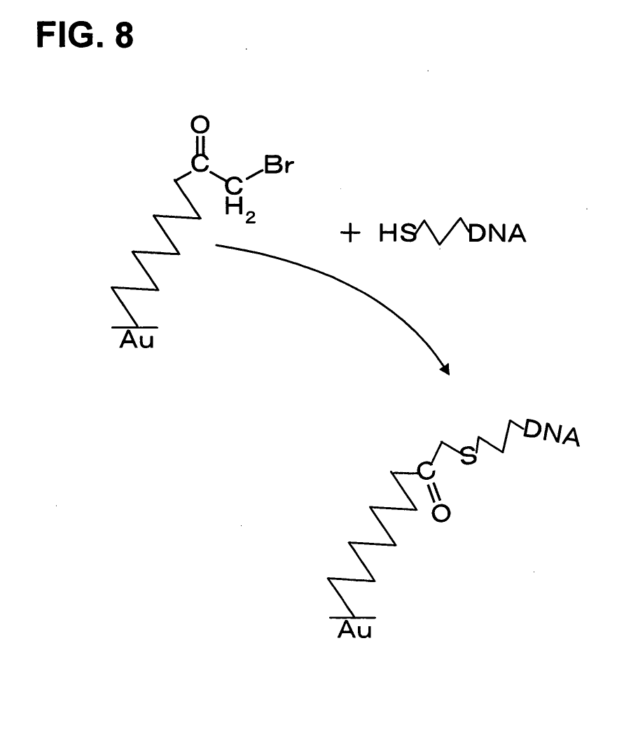

For example, an α-keto-amine is formed with a DNA having an amino group according to the route shown in FIG. 7, or an α-keto-thioether is formed with a DNA having a thiol group according to the route shown in FIG. 8.

An analyte evaluating device according to the present invention can be manufactured by subjecting a Au layer to any of the above-described reactions in an aqueous solution. It is to be noted that compounds having a thiol group on one end, and a carboxy group, thiol group, amino group, thioisocyanate group, isocyanate group, or α-keto halide group on the other, can be manufactured by known methods such as hydrolysis of an ester, reduction of a disulfide, reaction of an amino group and phosgene or reaction of an amino group and bis(trichloromethyl)carbonate, halogenation of a hydroxymethyl ketone or methanesulfonation. DNAs having an amino group or thiol group may be manufactured by known methods. DNAs having a maleimide group may be manufactured by the reactions such as one between a DNA having an amino group and EMCS(N-(6-maleimidecaproyloxy) succinimide) or HMCS(N-(8-maleimidecapryloxy) succinimide).

When such a carrier body is immersed in an aqueous solution, it is possible to expand the analyte binding part as a direct current electric field is, for example, applied between the carrier body and a counter electrode in the aqueous solution, and the analyte binding part shrinks spontaneously as the electric field is cut off.

Or, even if a counter electrode is absent, it is possible to make a negatively charged analyte binding part to expand by Coulomb repulsion, when a negative electric field is applied to the carrier body (electrode).

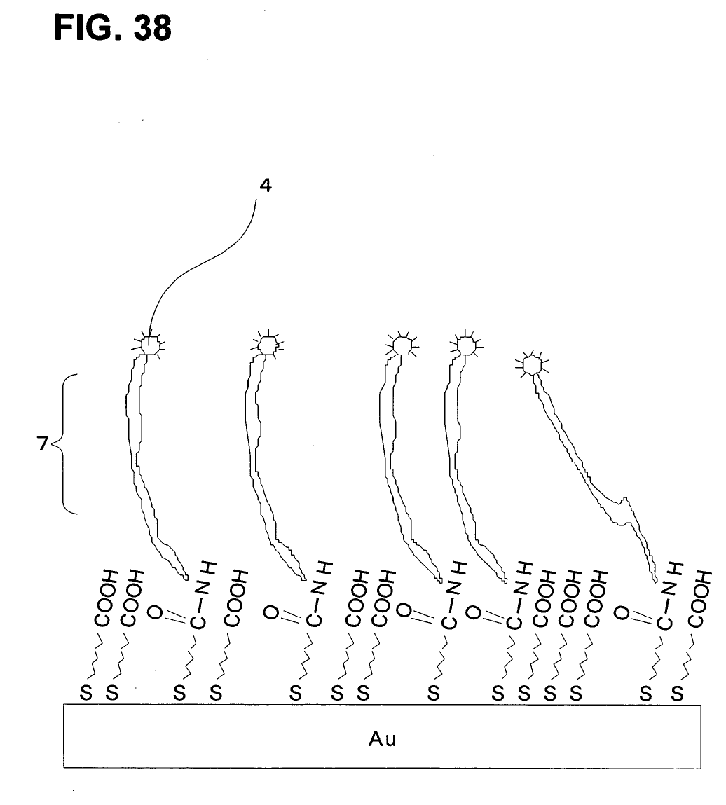

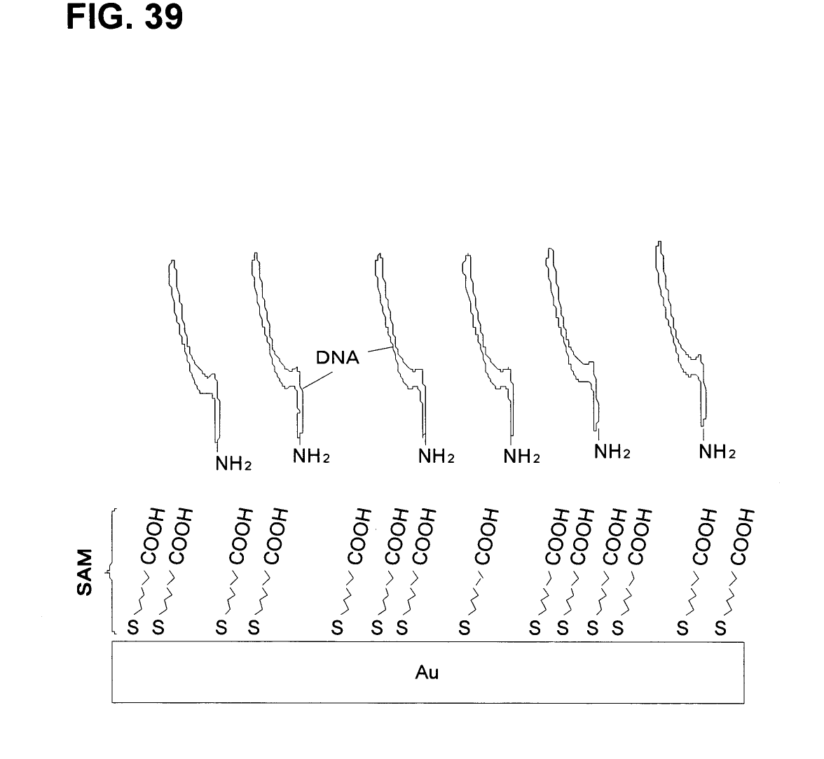

Therefore, when the analyte binding part is regarded as a responding part according to the present invention, a fluorescence-labeled part in the analyte bound to the analyte binding part can be made to emit and extinguish fluorescence. FIG. 38 illustrates an example.

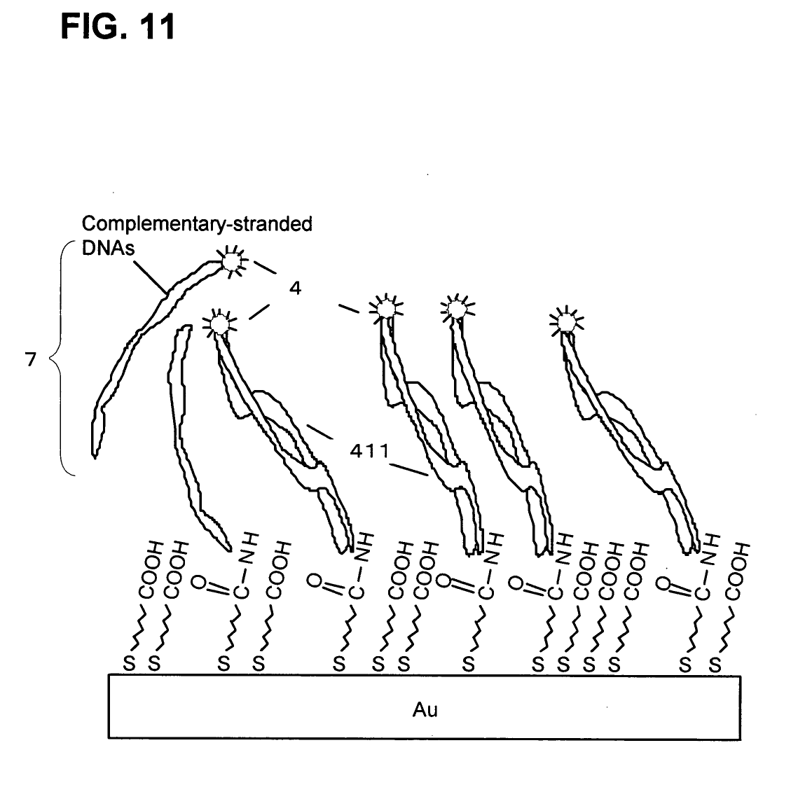

In addition, such an analyte binding part sufficiently serves for the purpose, as long as it can be finally bound with an analyte. Accordingly, it may have an intermediate part 411 in the intermediate section as shown in FIG. 11 that can be bound with both an analyte binding part and an analyte, for example.

As a result, a fluorescence-labeled part near the electrode that had extinguished fluorescence starts to emit fluorescence owing to the fact that the fluorescence-labeled part moves away from the surface of the electrode sufficiently.

Any known materials may be used as the light irradiation device for the fluorescence-labeled part such as a fluorescent molecule to emit fluorescence, and the fluorescence detecting device for detecting fluorescence emitted from the fluorescence-labeled part. Use of one or more optical fibers is advantageous in many cases, since the devices should be applied to a microscopic area. Optical fibers with an inner diameter of about 5 μm to about 1 mm can be used.

Laser light irradiation devices are often preferable, since they are applied to a microscopic area. Exciting fluorescent molecules by evanescent waves generated at the total reflection may be also advantageous, in order to adjust the light detection area which will be described later.

Installing a lens, a confocal lens in particular, between the light irradiation device and the carrier body is also advantageous when adjusting the light detection area.

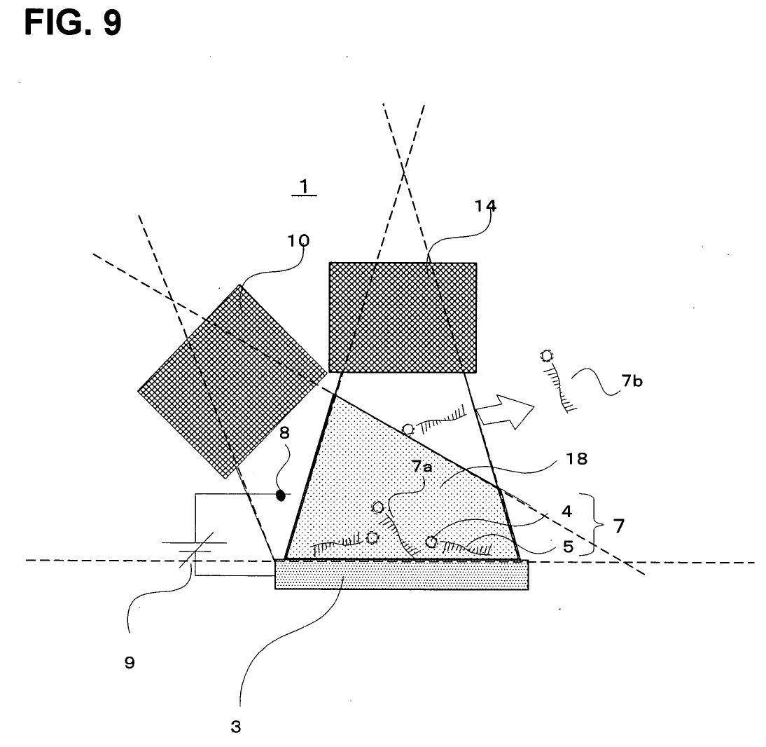

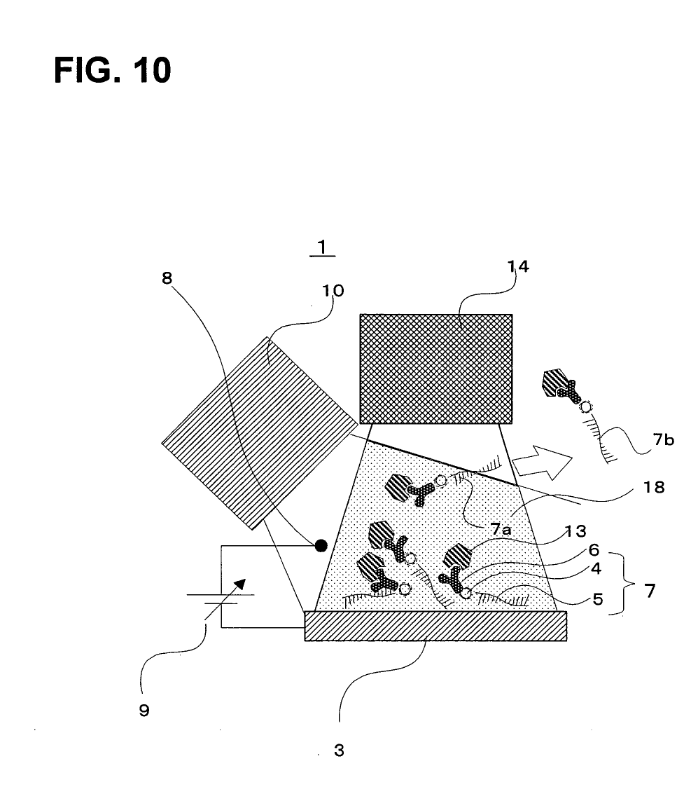

Next, a case is explained, using FIGS. 9 and 10 in which an electrode acts as a carrier body, an electromagnetic action is realized by providing a potential difference between the electrode and a counter electrode, and accordingly, analytes bound to a carrier body are detached from the carrier body, so that the fluorescence emitted as a result of the detachment is detected.

FIG. 9 is a model view illustrating that fluorescence is generated by irradiating light on analytes on the way of leaving the carrier body, and the fluorescence is detected. In FIG. 9, an analyte evaluating device 1 according to the present invention comprises an electrode 3 bound with analytes 7 having a fluorescence-labeled part 4 and a responding part 5, a counter electrode 8 in the aqueous solution, an external electric field applying device 9, a light irradiation device 10 comprising optical fibers, and a fluorescence detecting device 14 comprising optical fibers.

As shown in FIG. 9, by applying a potential difference between the electrode 3 and the counter electrode 8 with the external electric field applying device 9, the analytes 7 bound with electrode 3 are detached from the electrode 3. The increase and decrease of fluorescence during the course are detected with the fluorescence detecting device 14.

Hereupon, it is to be noted that FIG. 9 illustrates a case in which the analytes 7 themselves are the evaluation objects, and accordingly, evaluation object binding parts and evaluation objects bound with the evaluation object binding parts are not included in the figure. In contrast, FIG. 10 illustrates a case in which evaluation object binding parts 6 that is part of the analytes 7 are bound with evaluation objects 13.

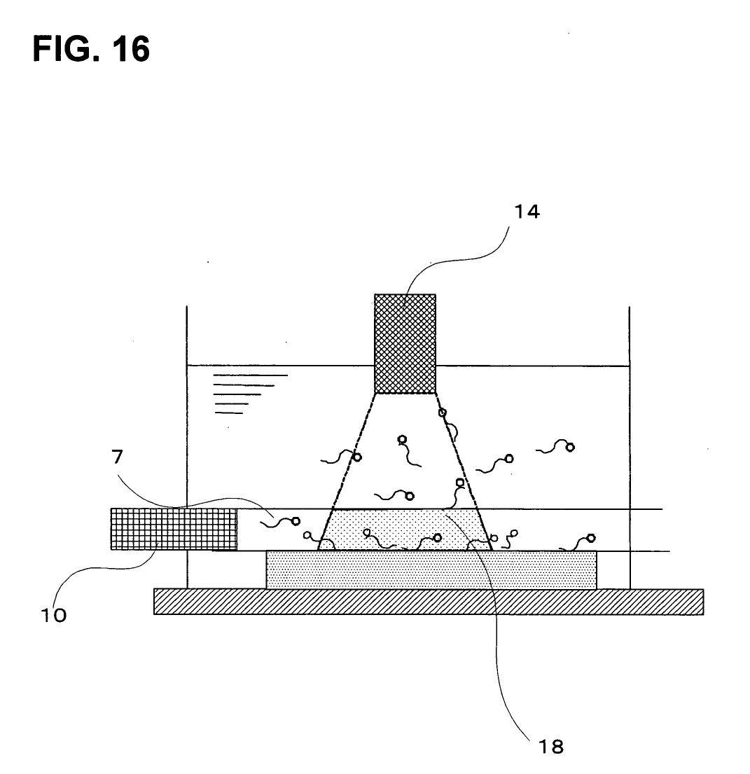

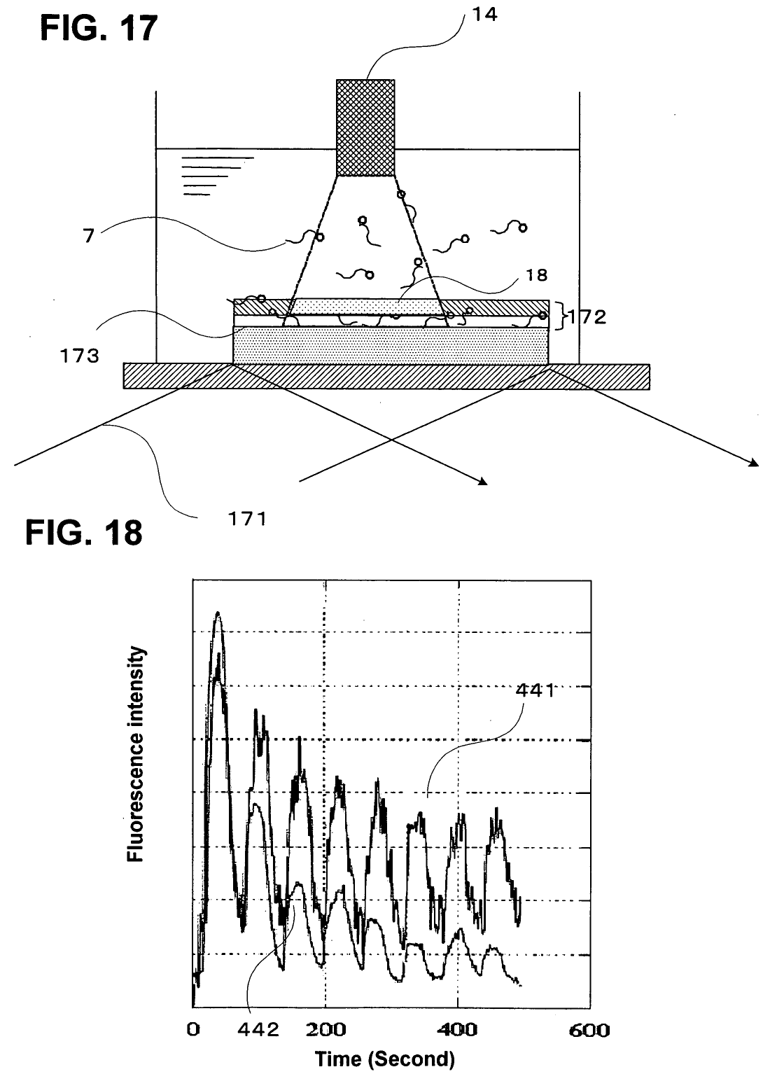

It is also to be noted that in the present invention, there are cases in which the fluorescence-labeled part near the carrier body can be excited, even when light is irradiated in a direction parallel with the surface of the carrier body as shown in FIG. 16, or when it is hard to say that light is irradiated on the surface of the carrier body as in the case of evanescent waves or the like, as shown in FIG. 17.

In such cases, the analytes are expanded or detached from the carrier body by electrostatic repulsion when an electric field with an appropriate polarity is applied. This expansion or detachment makes the fluorescence-labeled part 4 which was extinguished by the quenching effect, emit fluorescence and the fluorescence is detected by the fluorescence detecting device 14.

As time passes further, the analytes 7 move out of the light detection area 18 shown as a tetragon in FIG. 9, by diffusion in the aqueous solution after the expansion and detachment of the analytes, and the fluorescence comes to disappear. Analytes 7a exemplify analytes inside the light detection area 18, and analytes 7b exemplify analytes outside the light detection area 18.

Furthermore, any type of placement including placing a light irradiation device 10 and a fluorescence detecting device 14 in parallel as shown FIG. 14, can also be employed. FIG. 14 is an example in which a plurality of optical fibers 141 are unified as a bundle, for the light irradiation device 10 and/or fluorescence detecting device 14.

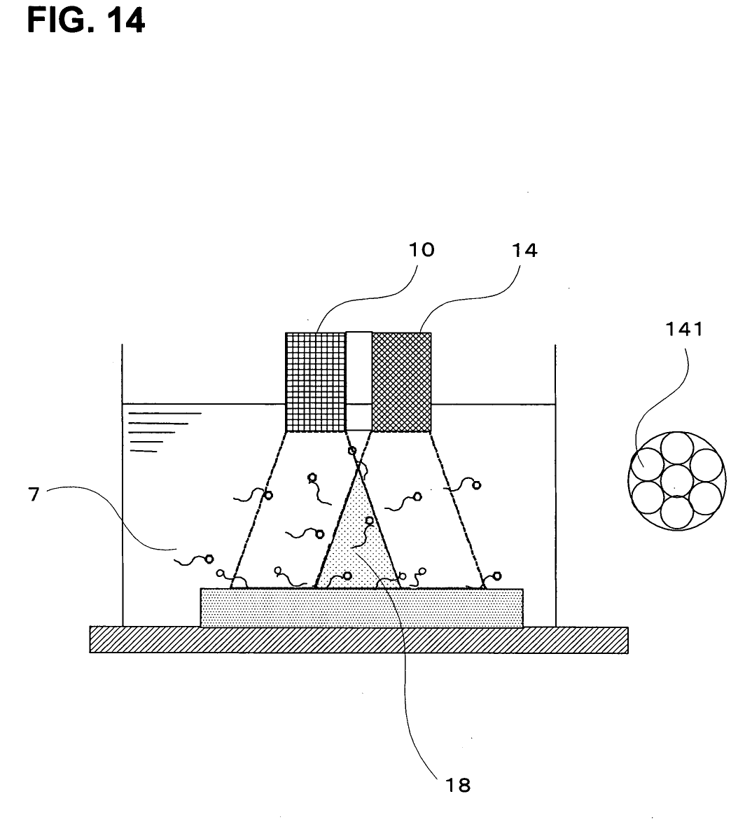

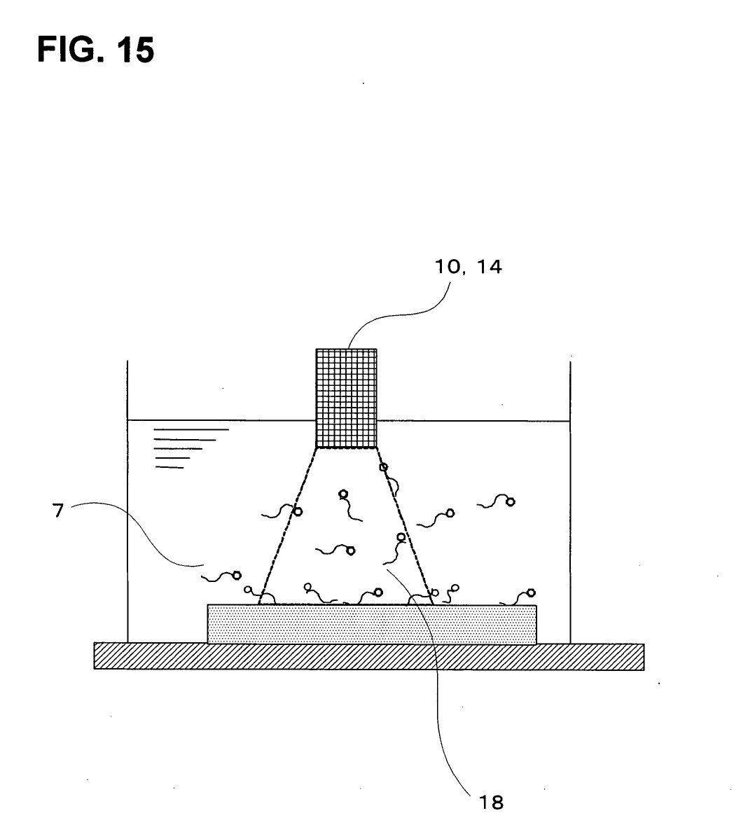

Regarding the installation of the light irradiation device 10 and fluorescence detecting device 14, exemplified are a device into which both are integrated, as shown in FIG. 15, a device in which the light irradiation device 10 is placed in parallel with the surface of the carrier body 3, as shown in FIG. 16, a device in which an evanescent field 172 to excite the fluorescence-labeled part 4 is generated by the totally-reflected light 171, as shown in FIG. 17, etc.

When a light irradiation device 10 is placed in parallel with the surface of the carrier body 3 as shown in FIG. 16, the thickness of the light detection area can be set as appropriately. For example, a thickness of 0.1 μm to 10 mm can be set. In addition, in the case shown in FIG. 17, the evanescent field can be thinned to an extreme thinness of not more than twice of the wavelength of light. Accordingly, it is also possible to make the fluorescence-labeled part extinguish fluorescence even when the analytes are not detached from the carrier body.

When the analytes are specifically bound with evaluation objects, the behavior of detachment and diffusion of the analytes naturally vary according to the masses and the electric charges of the evaluation objects. Accordingly, the fluctuation of the fluorescence intensity also varies greatly. Therefore, by utilizing such a fluctuation, it is possible to use the analyte evaluating device according to the present invention so as to detect evaluation objects such as proteins with a high sensitivity.

So far, explanation has been mainly made to an analyte evaluating device according to the present invention in which the carrier body is able to be bound with analytes and has not been bound yet. However, the analyte evaluating device according to the present invention is not limited to this, and those in which the carrier body has been already bound with analytes are also included in the category of the present invention.

Also, more than one carrier body can be used, with various types. Analyte evaluating devices comprising a plurality of carrier bodies of the same type or different types are also included in the category of the present invention.

Similarly, analyte evaluating devices comprising a plurality of analytes of the same type or different types are also included in the category of the present invention.

It is preferable that an analyte evaluating device according to the present invention is equipped with a plurality of carrier bodies, and each carrier body is provided with an electric potential different from those for the other carrier bodies, so as to make it possible for each carrier body to be bound with analytes of a different type. This way of use is advantageous in evaluating various types of analytes.

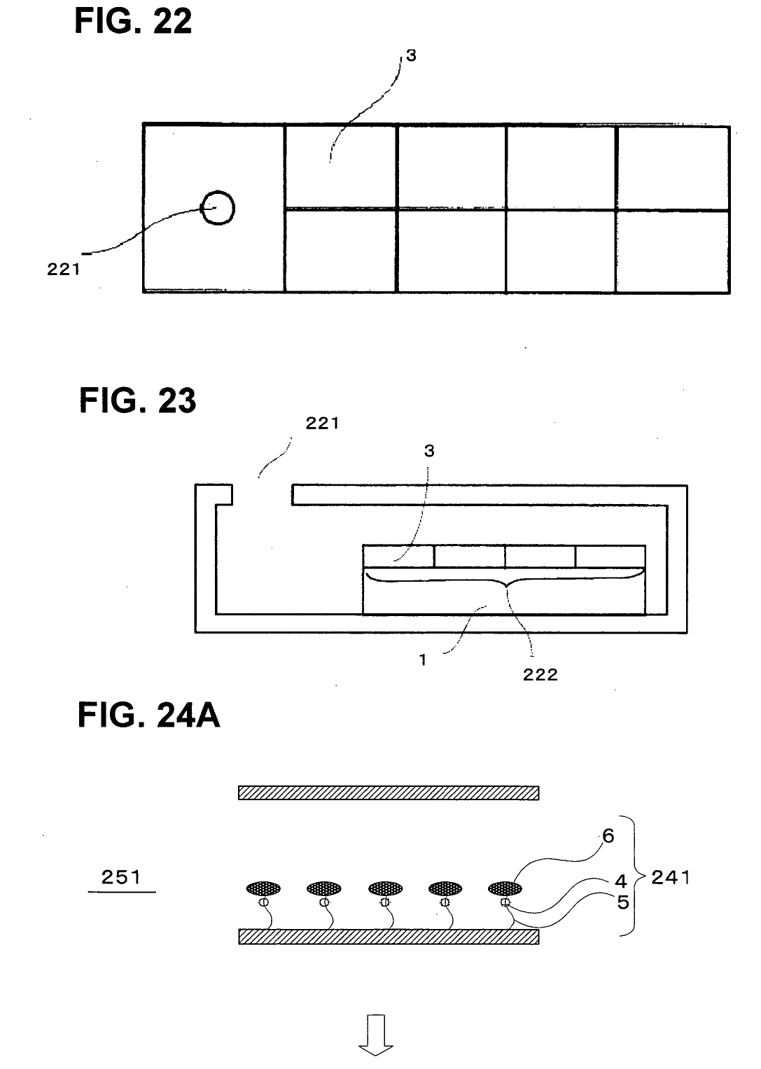

FIG. 22 illustrates a model plan view of a general type of biochip, and FIG. 23 illustrates a model cross-sectional side view thereof. When a sample is evaluated (qualitatively or quantitatively) using the biochip shown in FIGS. 22 and 23, it is a common procedure that the sample is introduced through a sample inlet 221 into a sensor array region 222 composed of multiple carrier bodies 3 installed on a substrate 1, where targets such as DNAs are captured by hybridization with analytes 7, and fluorescence signals are evaluated, utilizing the fluorescence-labeled parts.

According to the present invention, analytes are bound with specific carrier bodies of an analyte evaluating device such as a biochip, by utilizing the difference in the coulomb force when a potential difference is provided between the analyte solution and the carrier bodies. Accordingly, by simply introducing an analyte solution onto a substrate with a plurality of carrier bodies, it is possible to make the analytes adhere onto the specific carrier bodies, and not onto the other carrier bodies. Manual operations can be excluded. Hereupon, the term “analyte solution” may be a mass of analytes themselves in a liquid state. It can also include a solution in which analytes are diluted with a liquid medium. Dilution with a liquid medium may be preferable, since the concentration of analytes on a specific area of specific carrier bodies can be easily adjusted.

Installation of a plurality of analytes on respectively different carrier bodies can be accomplished as follows: every time an analyte solution containing a different type of analyte is used, an electric potential is provided to a carrier body for a particular purpose that is different from those provided to the other carrier bodies, when an electric potential difference is provided between the analyte solution and the carrier body, so that a specific analyte is electrically attached to a specific carrier body. It goes without saying that various reactions such as explained before can be utilized in this case. In many of these chemical reactions, the electric attachment is facilitated by the voltage regulation. Furthermore, selective installation may be possible by the help of voltage regulation, in many cases.

Also, regarding the analyte evaluating device according to the present invention, it is preferable that each of a plurality of carrier body installation sites is given an electric potential that is different from those for the other sites, so as to install different types of carrier bodies on respective carrier body installation sites, since similar effects can be accomplished.

Installation of respectively different carrier bodies on a plurality of carrier body installation sites can be accomplished as follows: every time a solution with a different type of carrier body is used, an electric potential is provided to a carrier body installation site for a particular purpose that is different from those provided to the other carrier body installation sites, when an electric potential difference is provided between the carrier body solution and the carrier body installation site, so that a specific carrier body is electrically attached to a specific carrier body installation site.

According to the analyte evaluating device of the present invention, the structure of the analyte evaluating device can be simplified, and miniaturization of the analyte evaluating device can be realized.

Also regarding a method for manufacturing an analyte evaluating device according to the present invention, it is preferable that each of a plurality of carrier bodies is given an electric potential that is different from those of the other bodies, so as to bind different types of analytes to respective carrier bodies, and that each of a plurality of carrier body installation sites is given an electric potential that is different from those of the other sites, so as to install different types of carrier bodies on respective carrier body installation sites. It is not always needed that these processes be accomplished only with the analyte evaluating device according to the present invention.

According to the method for manufacturing an analyte evaluating device according to the present invention, simplification of the structure of the analyte evaluating device is possible. It is possible to eliminate steps for fabricating complex shapes by photolithography or the like. Furthermore, manual installation works on specific sites can be abolished. Accordingly, shortened production time, simplified steps and easier handling of materials to be used can be realized. Miniaturization of the analyte evaluating device is also possible.

While installation of analytes must be performed before attaching a lid onto an analyte evaluating device in the case of the conventional methods for installing analytes, since in the conventional methods, installing analytes is not feasible after the attachment of a lid onto the analyte evaluating device, and accordingly, there is a fear that the biochip manufacturing steps after the analyte installation may degrade the analytes. However, by employing the installation method according to the present invention, it is only necessary to pour an analyte solution, and therefore, it is possible to make the analytes bound and installed at an arbitrary step after the production steps that may degrade the analytes. The problem of degrading the analytes can thus be eliminated. It is also possible to install analytes after an analyte evaluating device is completed. Similarly, it is also possible and useful to install different types of carrier bodies on respective installation sites after a lid is put on an analyte evaluating device.

In addition, as analytes can be easily installed on microscopic areas as well as areas having complex shapes that are difficult to physically access, freedom in designing an analyte evaluating device is increased. By this, it is possible to increase the number of carrier bodies that can be installed per a specific area, or to make an analyte evaluating device smaller than the conventional devices.

It is also possible to install devices and apparatus units having other functions together with an analyte evaluating device according to the present invention. For example, an analyte evaluating device comprising a flow path, an evaluation object capturing part to capture an evaluation object with a first capture body, and a capture body capturing part to capture a first capture body that has not captured an evaluation object, with a second capture body, in this order, can be used for evaluation by using an analyte evaluating device or by using a method for evaluating an analyte as explained heretofore, when the combined structure of the evaluation object and the first capture body is handled as an analyte.

Preferable are that the evaluation object capturing part is equipped with a carrier body that can be bound to or detached from the first capture body by the presence or absence of an external action, and that the capture body capturing part is equipped with a carrier body that can be bound to or detached from the second capture body by the presence or absence of an external action.

When the evaluation object capturing part is equipped with a carrier body that can be bound to or detached from the first capture body by the presence or absence of an external action, manufacture of the evaluation object capturing part becomes easier. It is also possible to make a first capture body that has captured an evaluation object and a first capture body that has not captured an evaluation object, detached from the carrier body under respectively different conditions, so as to easily separate them. Similar effects are possible for the capture body capturing parts.

As previously described, the presence or absence of an electromagnetic action or a chemical action can be utilized as the presence or absence of an external action. Specifically, the presence or absence of electromagnetic action is preferably generated by providing or by not providing a potential difference between an electrode and counter electrode.

In many cases, it is more preferable that a first capture body has a property to be specifically bound with an evaluation object, since only a specific evaluation object is captured by the evaluation object capturing part.

In addition, when a first capture body can be specifically bound with an evaluation object and a second capture body in the same sites, it can capture only a specific evaluation object, and the capturing site of the first capture body that has already captured the evaluation object has been plugged with the specific evaluation object. Accordingly, it becomes possible to prevent the first capture body from being bound to a second capture body. This will make it easier to differentiate first capture bodies that have captured evaluation objects from those that have not captured evaluation objects.

The relationship between an evaluation object and a first capture body is preferably the same as the relationship between an evaluation object and an evaluation object binding part or a responding part of an analyte in the above-described analyte evaluation device's main body. A first capture body may be fabricated by using materials that are similar to those for the above-described evaluation object binding part and the responding part. It is not always necessary for the first capture body to be able to expand and shrink. However in many cases it is more convenient for the evaluation on the analyte evaluating device's main body that it has such an ability.

The relationship between a first capture body and a second capture body is similar. A second capture body may be preferably fabricated by using materials that are similar to those for the above-described evaluation object binding part and the responding part. However, it is not necessary for the second capture body to have a fluorescence-labeled part, and to expand and shrink. Even if it is not detached from the capture body capturing part, it can serve for the purpose of the present invention.

In addition, both the first capture body and the second capture body can be fabricated, by using materials that are the same as those for the carrier body on the analyte evaluating device's main body.

Specific examples may be a case in which at least one of the first and second capture bodies has at least one type of group selected from the class consisting of a carboxy group, thiol group, amino group, thioisocyanate group, isocyanate group and α-keto halide group; a case in which the first capture body can be bound with an evaluation object by one of the following reactions; a case in which the second capture body can be bound with the first capture body by one of the following reactions; or a case in which both of the last two cases are combined by one of the following reactions.
A. a reaction between a carboxy group and an amino group via an imidazole-bound intermediate that is activated by 1-(3-dimethylamino-propyl)-3-ethyl-carbodiimide hydrochloride,
B. a reaction between a carboxy group and an amino group via an N-hydroxysuccinimide-bound or an N-hydroxysuccinimide sulfonic acid-bound intermediate that is activated by 1-(3-dimethylamino-propyl)-3-ethyl-carbodiimide hydrochloride,
C. a reaction between a thiol group and a maleimide group,
D. a reaction between an isocyanate group and an amino group,
E. a reaction between an α-keto halide group, and an amino group or thiol group;

In such cases, it is preferable that the carrier body of the evaluation object capturing part has a Au layer on the surface, and the first capture body can be bound with or detached from the Au layer via a thiol group; and that the carrier body of the capture body capturing part has a Au layer on the surface, and the second capture body can be bound with or detached from the Au layer via a thiol group.

When there are a carrier body to bind the first capture body to the evaluation object capturing part, and/or a carrier body to bind the second capture body to the capture body capturing part, the same materials for the carrier body of the analyte evaluating device's main body, can be used for the carrier bodies. It is not always necessary for the carrier bodies to be fixed, and any shape can be accepted as long as it complies with the gist of the present invention. For example, a design in which first and second capture bodies are installed on a carrier body in a granular form, and bottle-neck sections are installed at the outlets of the evaluation object capturing part and/or capture body capturing part so as to make the carrier body particles get stuck at the bottle-neck sections, thus preventing the first and second capture bodies from flowing out of the sites, is preferable to facilitate various kinds of separation.

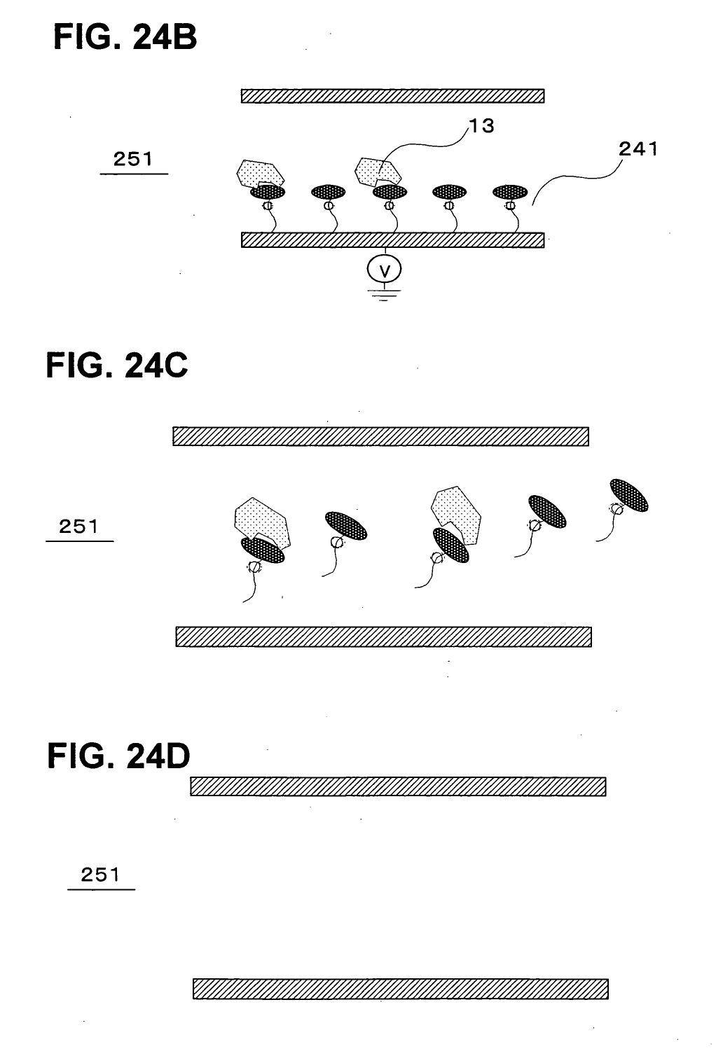

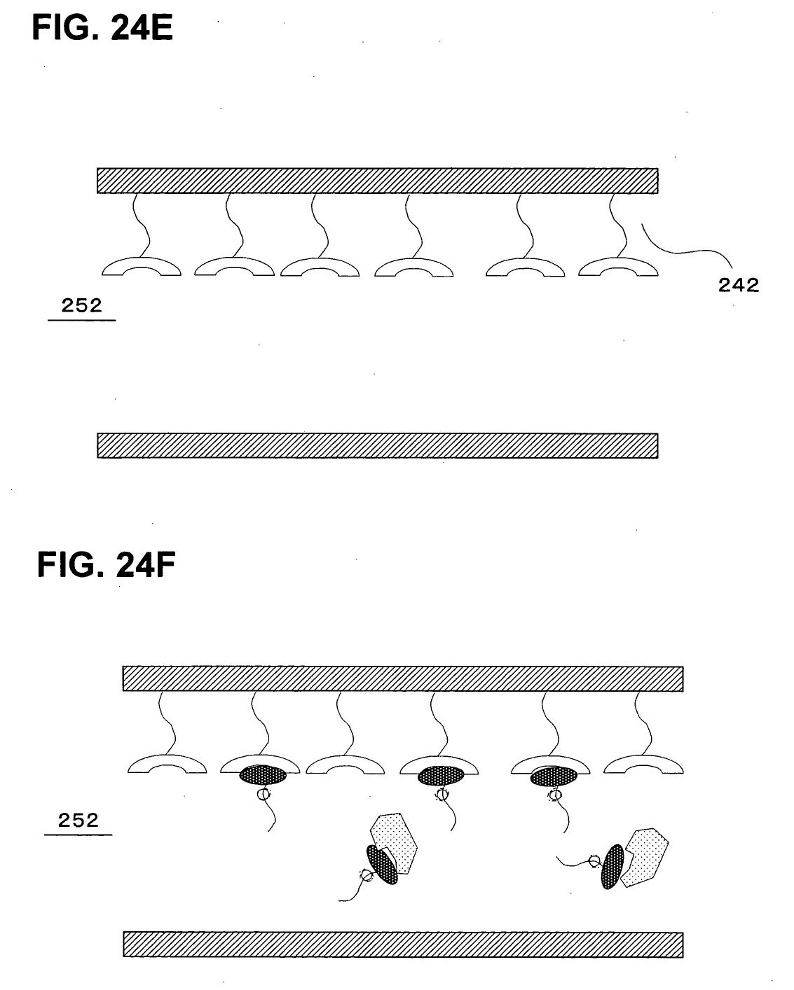

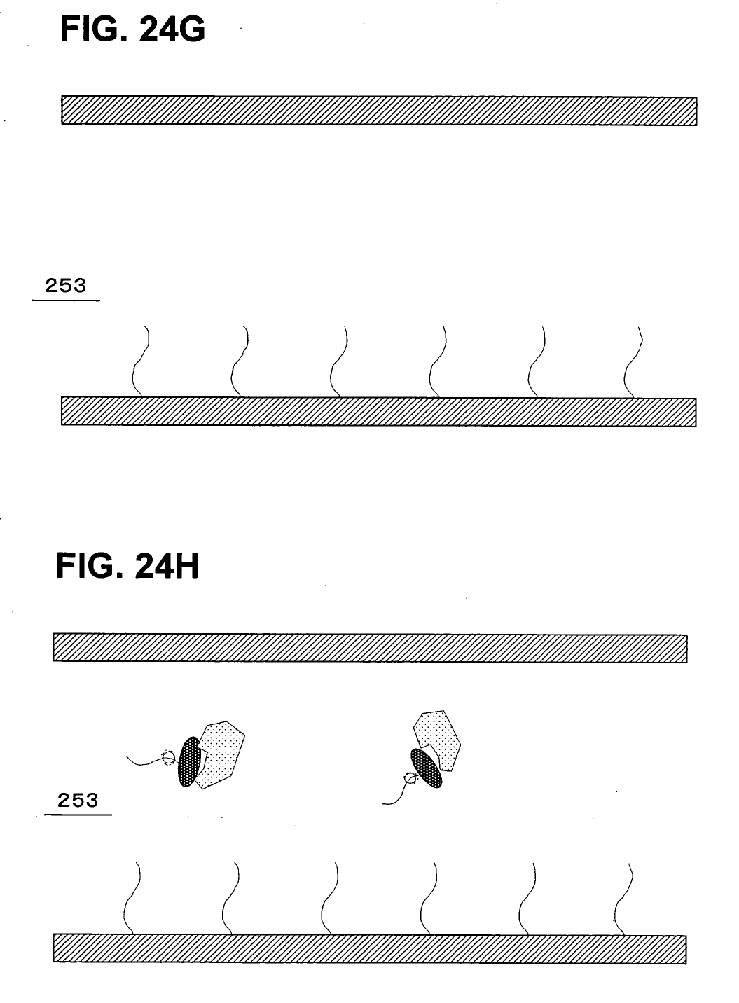

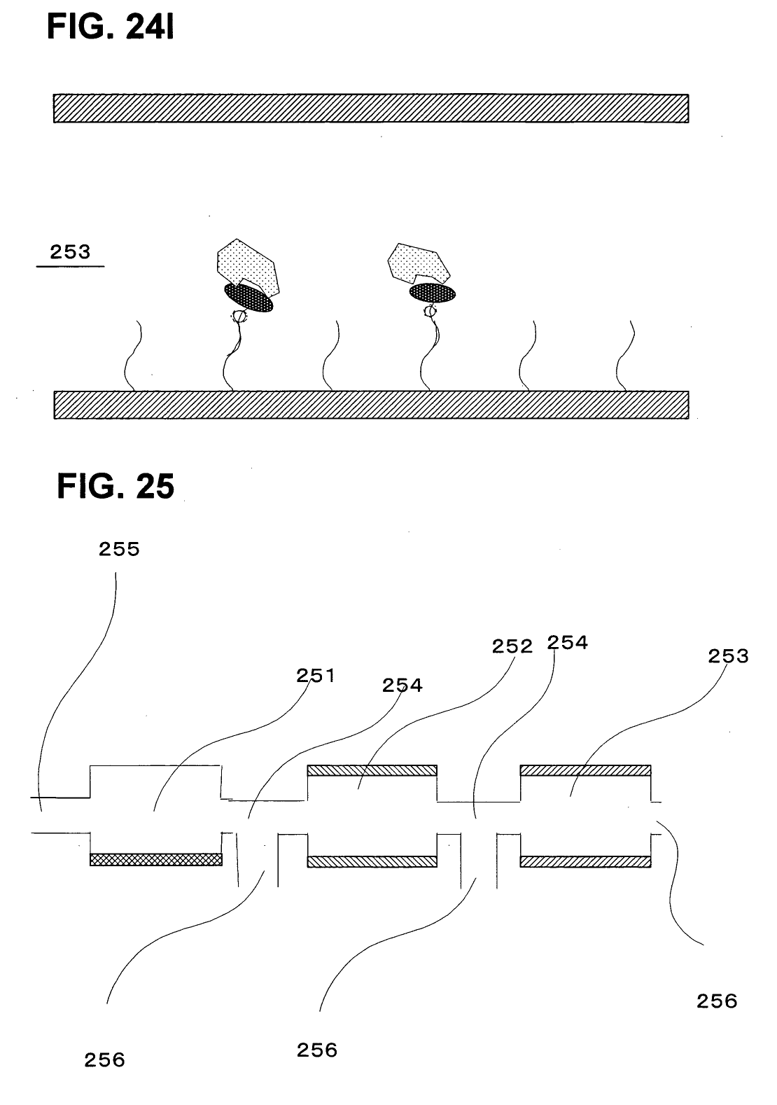

Evaluation using an analyte evaluating device having the above-described evaluation object capturing part and capture body capturing part installed in this order is illustratively explained, using FIGS. 25 and 24A to 24I. FIG. 25 is a model cross-sectional view illustrating that an analyte evaluating device 1 according to the present invention has an evaluation object capturing part 251, a capture body capturing part 252 and an analyte evaluating device's main body 253 that are connected with flow paths 254. In the figure, numeral 255 indicates a sample inlet and numeral 256 indicates a sample outlet. FIGS. 24A to-24I are model views illustrating how the capturing and detachment occur in the combination of the evaluation object capturing part 251, capture body capturing part 252 and analyte evaluating device's main body 253.

The principle of the operation is as follows. First, a solution of evaluation objects is poured into the evaluation object capturing part 251 that is bound with the first capture bodies 241 in FIG. 24A for the first capture bodies 241 to electrostatically capture the evaluation objects 13 as shown in FIG. 24B, by applying a specific potential difference, for example. In this example, a fluorescence-labeled part 4, a responding part 5, and an evaluation object binding part 6 are installed on the first capture body.

Next, first capture bodies 241 that are bound with evaluation objects 13 as well as first capture bodies 241 that are not bound, are released into the medium in the flow path by the electromagnetic or chemical action such as, for example, electrostatic repulsion or chemical scission, as shown in FIG. 24C. Since the evaluation object capturing part 251 comes to be in a state of being not bound with first capture bodies in this stage, as shown in FIG. 24D, it can be reused by binding new first capture bodies.

After that, this solution is introduced into a capture body capturing part 252 having second capture bodies 242 that are to be specifically bound with first capture bodies that have not been bound with evaluation objects, as shown in FIG. 24E, to perform capturing as shown in FIG. 24F. This capture body capturing part 252 can be reused by detaching the captured first capture bodies 241 by some means or other, or by detaching the captured first capture bodies together with the second capture bodies 242, followed by installation of new second capture bodies, or by detaching the captured first capture bodies together with the carrier body, followed by installation of a new carrier body, or the like.

Accordingly, only the first capture bodies that are bound with the evaluation objects are introduced into the analyte evaluating device's main body. It is possible to install a carrier body on the analyte evaluating device's main body 253 that can be bound with the first capture bodies that are bound with the evaluation objects, as shown in FIG. 24G.

When the first capture bodies that are bound with the evaluation objects are introduced into the analyte evaluating device's main body 253 as shown in FIG. 24H, they are bound with the carrier body as shown in FIG. 24I. Accordingly, the presence or absence of an evaluation object, quantitative analysis thereof, etc. can be performed by making the fluorescence-labeled parts 4 of the first capture bodies emit fluorescence by irradiating with light.

In this way, it is possible to perform evaluation, without modifying the evaluation object with a fluorescent molecule or by radiation labeling.

Furthermore, when the first capture bodies that are captured as being not bound with evaluation objects as shown in FIG. 24F, are detached later, and introduced into the analyte evaluating device's main body 253, it is possible to detect the first capture bodies that are not bound with evaluation objects. This embodiment belongs to the present invention, too.

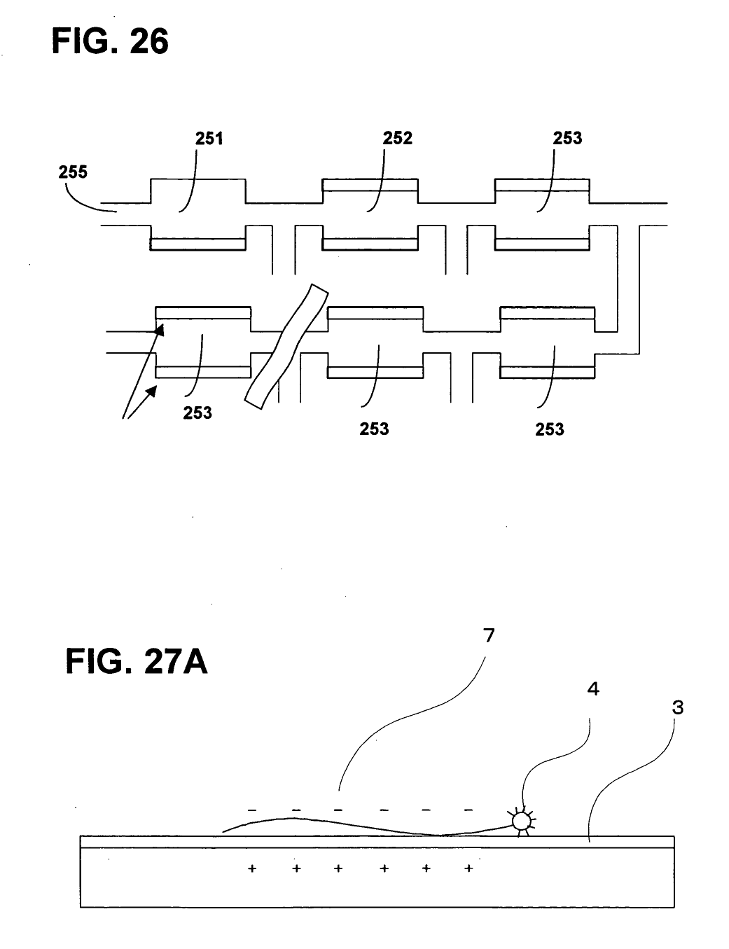

The above explanation was made on a case in which one type of evaluation object is involved. However, it is also possible to evaluate plural types of evaluation objects, by employing plural types of first capture bodies in an evaluation object capturing part 251, disposing second capture bodies that correspond to the respective first capture bodies in a capture body capturing part 252, and connecting, in series, plural analyte evaluating device's main bodies 253 that correspond to the respective evaluation objects as shown in FIG. 26. In such a case, it is preferable to connect another analyte evaluating device's main body 253′ to use it as a reference that does not interact with any of the evaluation objects.

Also, the above explanation was made on a case in which the analyte evaluating device's main body comprises a carrier body that can be bound with an analyte having a fluorescence-labeled part that can emit fluorescence by light received when the distance between the fluorescence-labeled part and the carrier body is enlarged, and a responding part with a distance between the fluorescence-labeled part and the carrier body that is variable by an external action, a light irradiation device for the fluorescence-labeled part to emit light, and a fluorescence detecting device for detecting the fluorescence emitted by the fluorescence-labeled part. However, devices combining the above-described evaluation object capturing part and capture body capturing part are useful not only in the above-described case but also in cases to separate a specific evaluation object from other materials. That is, an analyte evaluating device having a flow path, an evaluation object capturing part for capturing an evaluation object with a first capture body, and a capture body capturing part for capturing a first capture body that has not captured an evaluation object, installed in this order, is possible and useful, when the analyte evaluating device's main body employs any known evaluation means such as a means using direct separation followed by evaluation using chemical, biochemical and similar other procedures, and a means measuring radiation amount, electric current, or intensity of various emissions.

According to the present invention, it is possible to perform evaluation without introducing a fluorescence-labeled part or a radioactive material into an evaluation object. Evaluation for a tiny amount of sample is possible. Evaluation at a high sensitivity is also possible. Furthermore, evaluation is possible even when multiple kinds of evaluation objects are present in a sample. Miniaturized, complex, and integrated analyte evaluating devices, analyte evaluation methods therefor, and methods for manufacturing the analyte evaluating devices can also be provided.

The analyte evaluating device realized by the present invention can be used, as a protein detecting device to see that part of a series of protein interaction networks from an insulin acceptor to a glycogenase is decreased or increased, for example, when the hepatic cell changes the intracellular glycogen metabolism, responding the reception state of insulin in diabetes mellitus.

Accordingly, by using such a protein detecting device, it is possible to comprehend the population of proteins, including so-called post-translational modifications such as phosphation and glycosilation.

In addition, it is possible, for example, to see that a functional degradation of a specific protein in relation with the interaction network is the cause of the defective glucose metabolism, instead of the conventional approach to see a phenomenon appearing as symptoms as a whole and correlate it with diabetes mellitus. This will make it possible to provide an appropriate diagnosis and treatment corresponding to the cause of the functional incompetence, and an appropriate verification of the result of treatment. Beside diabetes mellitus, the same procedure may be applicable to high blood pressure, hyperlipidemia, cancer (imperfect cell growth control) and other multifactorial diseases in general.

### EXAMPLES

The present invention is further explained in reference to the following examples.

### Example 1

An oligonucleotide (analyte) bound with a monoclonal IgG antibody (evaluation object binding part) and a fluorescence molecule was physically adsorbed on a Au electrode (carrier body) formed on a sapphire substrate. An antigen (protein) as an evaluation object was poured sequentially so that the poured amounts were 0.1 fM, 0.2 fM, 0.5 fM, and 1.0 fM, so as to be bound with the monoclonal IgG antibody (evaluation object binding part) of the oligonucleotide. The time changes of the respective fluorescences were measured using the arrangement shown in FIG. 9.

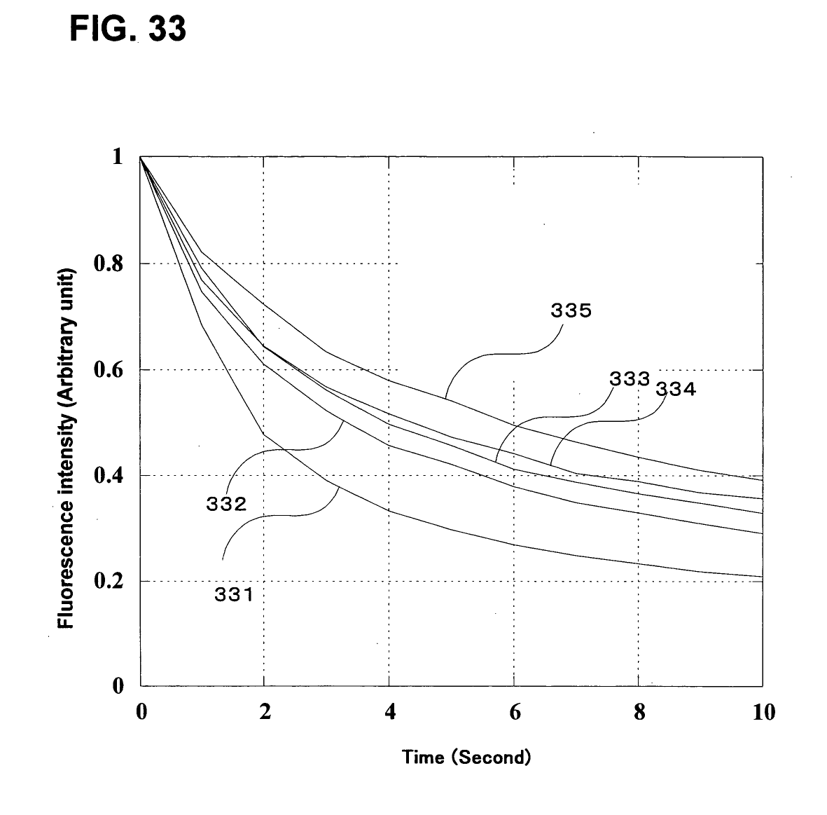

A negative electric field of −500 mV was applied to the electrode. FIG. 33 shows the result. Numeral 331 indicates 0 fM, numeral 332, 0.1 fM, numeral 333, 0.2 fM, numeral 334, 0.5 fM, and numeral 335, 1.0 fM. The result indicates that a supermicroscopic amount of a protein to be detected on a sub fM order can be quantitatively detected.

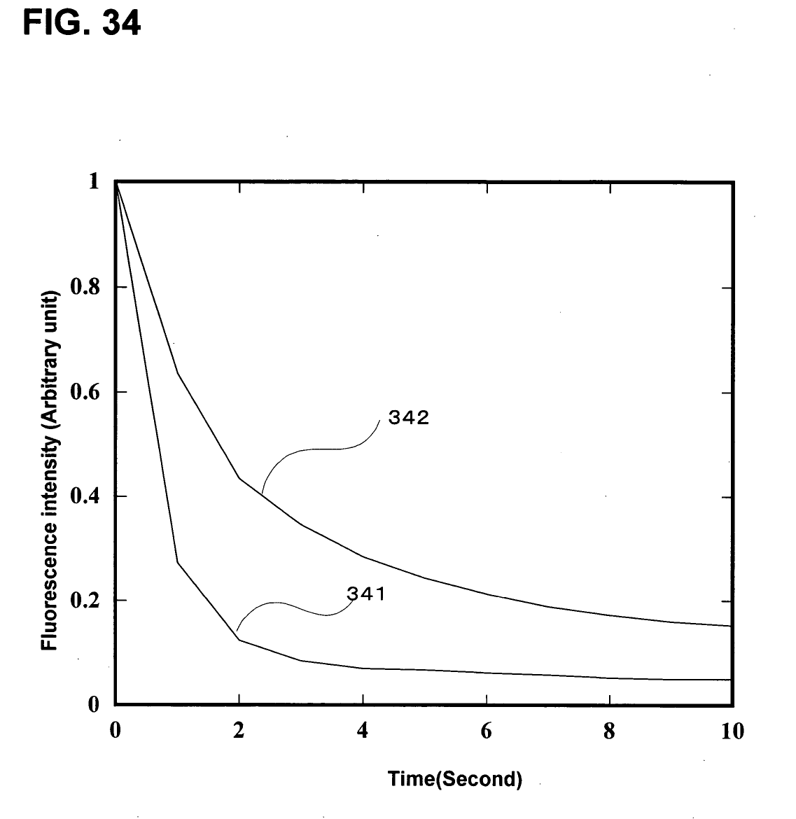

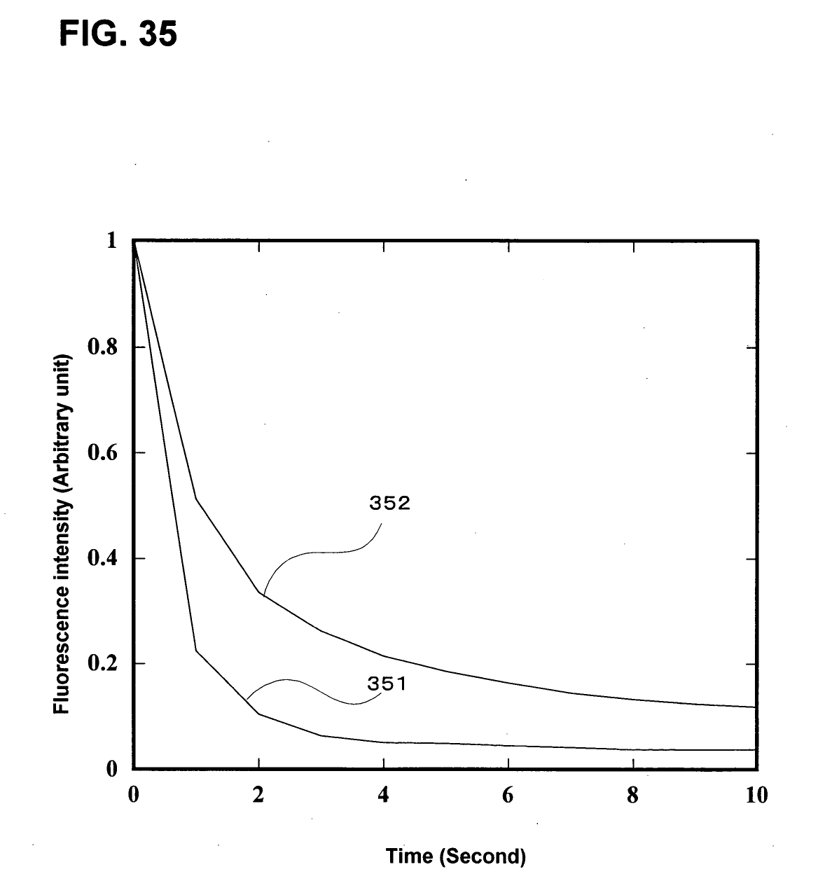

FIGS. 34 and 35 show the results when the measurement was performed in arrangements as shown in FIGS. 16 and 17 so as to further raise the measurement precision. Numerals 341 and 351 indicate 0 fM, and numerals 342 and 352 indicate 0.1 fM. When these graphs are compared with FIG. 33, the decay of fluorescence intensity at 0.1 fM is smaller than 0 fM in FIGS. 34 and 35. From this, it is understood that the arrangements in FIGS. 16 and 17 can provide detection with a higher precision.

It is possible to quantitatively analyze an evaluation object such as a protein by observing a phenomenon such as the above. Hereupon, it is to be noted that the detection sensitivity of an evaluation object such as a protein varies, depending on the molecular weight of the evaluation object to be bound as well as a binding constant between an evaluation object and an evaluation object binding part (for example, monoclonal IgG antibody). Therefore, a wider range of measurement can be covered, for example, by arranging a plural number of monoclonal antibodies with different binding constants on an array.

### Example 2

A solution of a single-stranded oligonucleotide bound with a fluorescence molecule and biotin was poured from the sample inlet 255 of FIG. 25, and kept in the evaluation object capturing part 251 for 5 hours, so as to make the single-stranded oligonucleotide (first capture body) bound with the fluorescence molecule and the biotin physically adsorbed on the Au electrode (carrier body) formed in the evaluation object capturing part 251. After that, the residual solution was poured out of the outlet 256.

Next, solutions of avidin (evaluation object) (0.1 fM, 0.2 fM, 0.5 fM, 1.0 fM, 5.0 fM, and 10 fM) were poured from the sample inlet 255, kept in the evaluation object capturing part 251 for 10 minutes, so as to react the avidin with the biotin. After that, the residual solution was poured out of the outlet 256.

Next, a solvent (physiological saline) is poured into the evaluation object capturing part 251. Then, the adsorbed first capture bodies were brought into the solvent, by applying a potential (−0.5 V) to the electrode of the evaluation object capturing part 251. The adsorbed first capture bodies included both first capture bodies that had captured evaluation objects and first capture bodies that had not captured an evaluation object.

Next, the medium that took in the first capture bodies was moved to the capture body capturing part 252 and kept there for 10 minutes, so as to make the first capture bodies that had not captured an evaluation object bound, by the biotin, to the second capture body (avidin in this case) fixed on the capture body capturing part 252, with a result that the first capture bodies that had not captured an evaluation object were separated from the first capture bodies that had captured evaluation objects.

The first capture bodies that had captured evaluation objects were unable to be bound with avidin as the second capture body, since the biotin had already been bound with avidin of the evaluation object. That is, this is an example of an analyte evaluating device in which a first capture body can be specifically bound with an evaluation object and a second capture body at the same site.

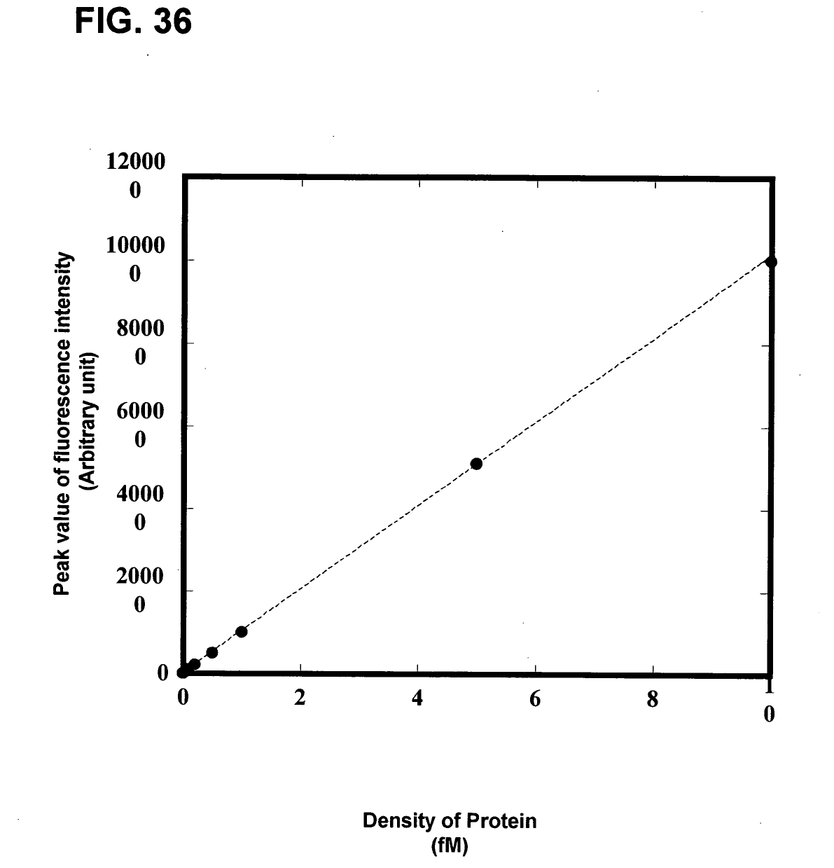

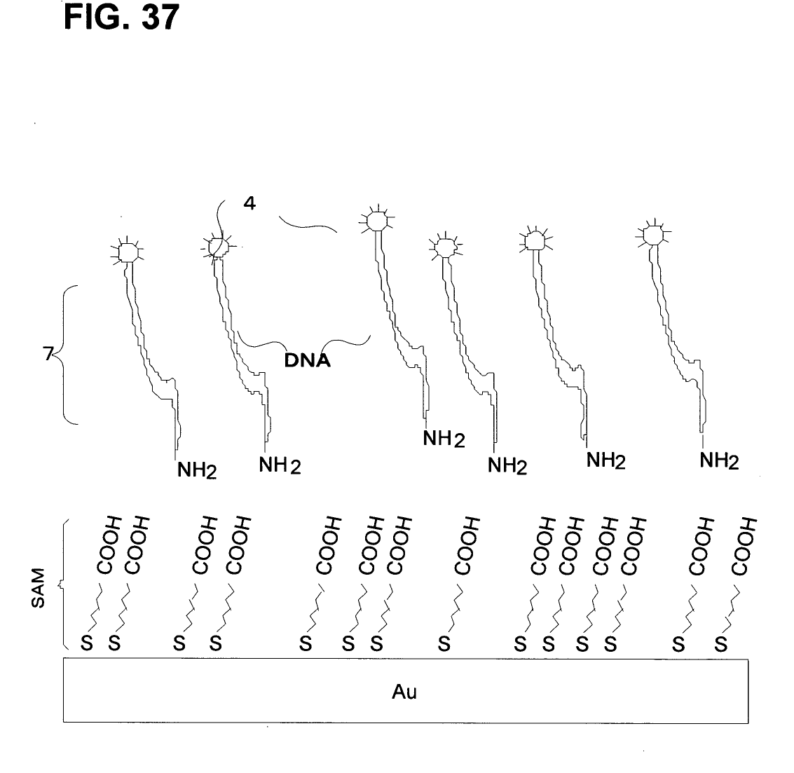

Next, this solution was introduced into an analyte evaluating device's main body 253, where the fluorescence from the fluorescence molecules was measured by irradiation from a laser. As the analyte evaluating device's main body 253, the device shown in FIG. 9 was used. A negative potential of −0.5 V was applied to the electrode to evaluate the fluorescence intensity (a peak value). The result is shown in FIG. 36. The result indicates that a supermicroscopic amount of the protein to be detected on a sub fM order can be quantitatively detected.

### Example 3

A Au electrode (carrier body) modified with a SAM formed by using molecules having a thiol group (—SH) and a carboxy group at both ends of the molecular chain, respectively, was used so as to be bound with DNAs (analytes) with an amino group according to the above-described method A, while activating the carboxy group, with an voltage applied or not applied.

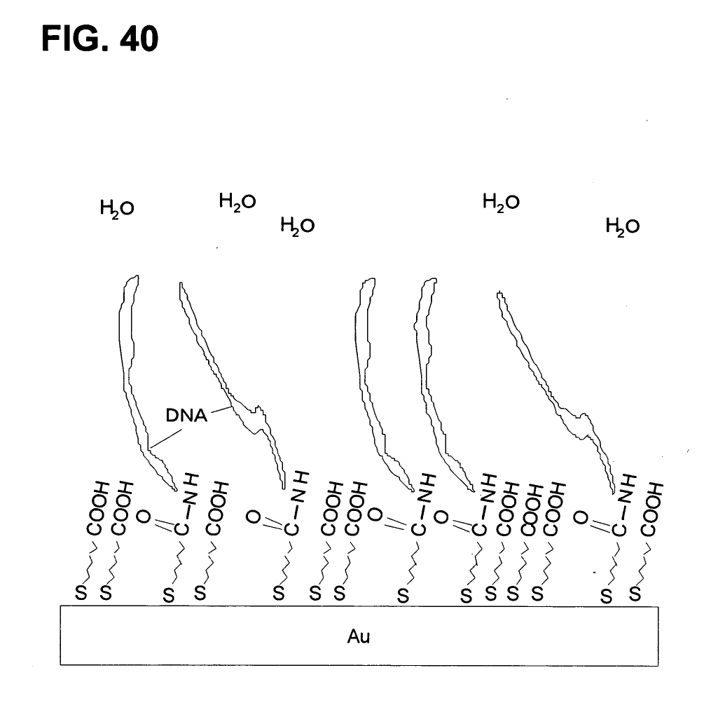

As the DNAs with an amino group, single-stranded DNAs (FIGS. 37 and 38) and double-stranded DNAs (FIGS. 39, 40 and 11) were used. FIG. 37 illustrates a state of single-stranded DNAs (analytes) having a fluorescent pigment 4 being in the vicinity of the SAM of the carrier body, and FIG. 38 illustrates a state of the amino groups of the single-stranded DNAs (analytes) having a fluorescent pigment 4 being bound with carboxy groups of the SAM to form amide bonding. FIG. 39 illustrates a state of single-stranded DNAs without a fluorescent pigment being in the vicinity of the SAM of the carrier body, and FIG. 40 illustrates a state of the amino groups of the single-stranded DNAs without a fluorescent pigment being bound with the carboxy groups of the SAM to form amide bonding. FIG. 11 illustrates a state of single-stranded DNAs having a fluorescent pigment 4 being hybridized with single-stranded DNAs without a fluorescent pigment that are bound with the carboxy groups of the SAM so as to form analytes.

A solution of an aliphatic carboxylic acid (with two or more carbon atoms) having a thiol group (—SH) in ethanol was introduced onto a polished circular Au electrode with a diameter of 1.6 mm, and allowed to react at room temperature for 24 hours. The thiol group reacted with the Au surface to form Au—S binding. A SAM was formed accordingly. The thiol group can be located on any position of the carboxylic acid. The number of carbons in an aliphatic carboxylic acid with a thiol group (—SH) is preferably two or more. Five or more is more preferable.

Onto this membrane, DNAs with an amino group were introduced to be bound via the route shown in FIG. 3. In the case of single-stranded DNAs, a fluorescent pigment 4 was introduced as shown in FIGS. 37 and 38. In the case of double-stranded DNAs, single-stranded DNAs with an amino group and without a fluorescent pigment were first introduced as shown in FIGS. 39 and 40, and then, single-stranded DNAs with a fluorescent pigment 4 and without an amino group that were complementary to the first single-stranded DNAs with an amino group and without a fluorescent pigment, were introduced as shown in FIG. 11. The fluorescent pigment can be introduced at either of the ends or at the 5′ terminal of the strand of the DNAs.

Specifically, a solution of DNAs having an amino group and an activation catalyst EDC solution were added to an imidazole buffer solution so as to react the DNAs having an amino group with the carboxy groups, in the course of which a potential was applied to the Au electrode to facilitate the reaction between the carboxy groups and the amino groups. When double-stranded DNAs were used, complementary DNAs were reacted after this step to form the double-stranded DNAs.

In this way, when the fluorescent pigment is activated by light, it extinguishes fluorescence in a state near the metal surface, and emits fluorescence when sufficiently distant from the surface.

The electrode with DNA strands formed on the SAM according to the above-described structure was immersed into an aqueous solution, a direct-current electric field or alternate-current electric field with modulations such as pulses was applied to the DNA strands by means of the two-electrode method. The fluctuation of the fluorescence intensity was measured, as the fluorescent pigments on the DNA strands were activated with a UV lamp to emit fluorescence. A fluorescence emitting phenomenon was experimentally ascertained in which fluorescence that had been extinct started to be emitted when an electric field was applied. It is to be noted that the three-electrode method can be employed instead of the two-electrode method.

Furthermore, the effect of the presence of avidin (a kind of protein) on the fluorescence intensity fluctuations was measured by introducing biotin (a kind of a coenzyme) at the tips of DNA strands.

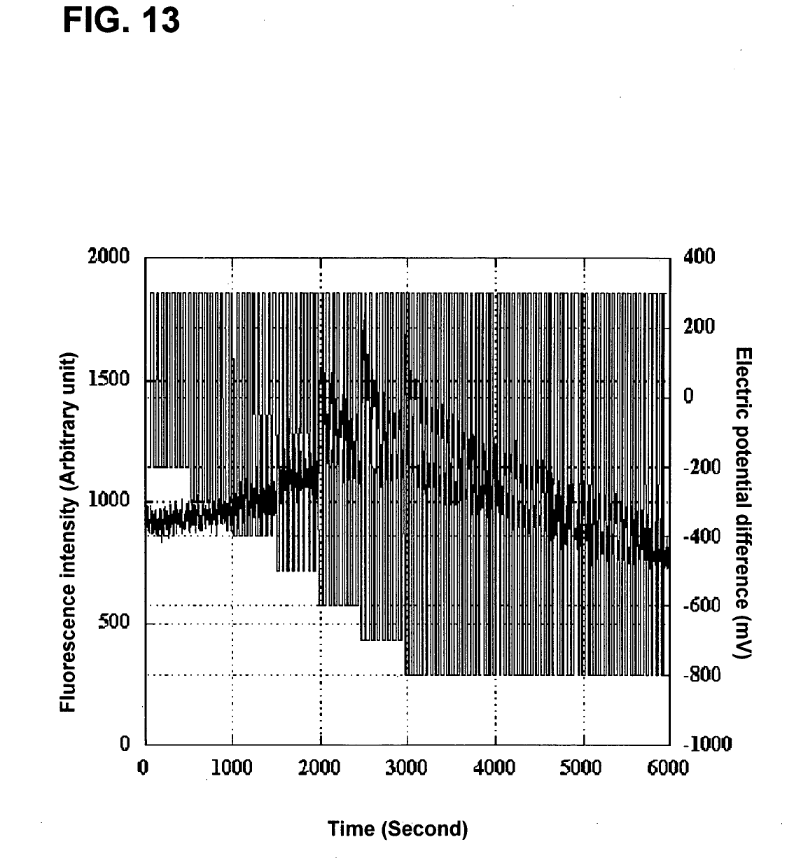

FIG. 13 illustrates the change of fluorescence intensity with time, when the level of a pulse voltage was changed in a stepwise manner, in a case in which a fluorescent pigment and biotin (evaluation object binding part) are introduced onto single-stranded DNAs, and then the biotin is bound with avidin (evaluation object) (with-protein case).

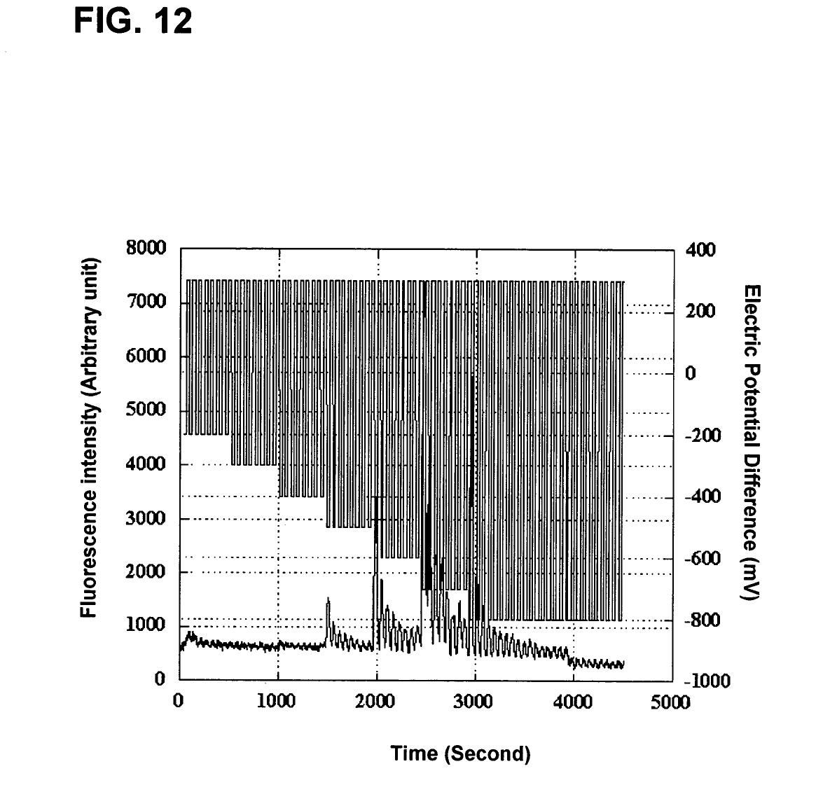

In contrast, FIG. 12 shows a change of fluorescence intensity with time when the level of a pulse voltages is changed step by step in a case in which a fluorescent pigment and biotin (evaluation object binding part) are introduced onto single-stranded DNAs, and avidin (evaluation object) as a protein is not bound with the biotin (without-protein case).

FIG. 18 shows a comparison of the changes of fluorescence intensity during 2,000-2,500 seconds in FIGS. 12 and 13. From the comparison of the fluorescence intensity of the with-protein case 441 with that of the without-protein case 442 in this graph, it is understood that the change of fluorescence intensity is made smaller when avidin is present, that is, that the decaying rate between each peak and the peak immediately following the peak is made smaller. It is possible to evaluate the presence or absence of a particular protein, the concentration, etc., from such a difference in behavior.

It is to be noted that fine up-and-down fluctuations of the fluorescence intensity is considered to be caused by repetition of motions that the fluorescent molecules bound with DNA strands emit light when moving away from the electrode, and extinguish light when they are attached to or come near the electrode, the motions being derived from the repetitive phenomenon that the DNA strands are expanded when a negative electric field is applied, and are shrunk spontaneously when the field is put off, and/or that the DNA strands with a negative charge are dissociated from the electrode by Coulomb repulsion when a negative electric field is applied to the electrode, and are re-attached to the electrode when the field is put off.

### Example 4

A biochip was prepared that had a sensor array part 222 as shown in FIGS. 22 and 23. The carrier bodies 3 in FIGS. 22 and 23 were made of Au.

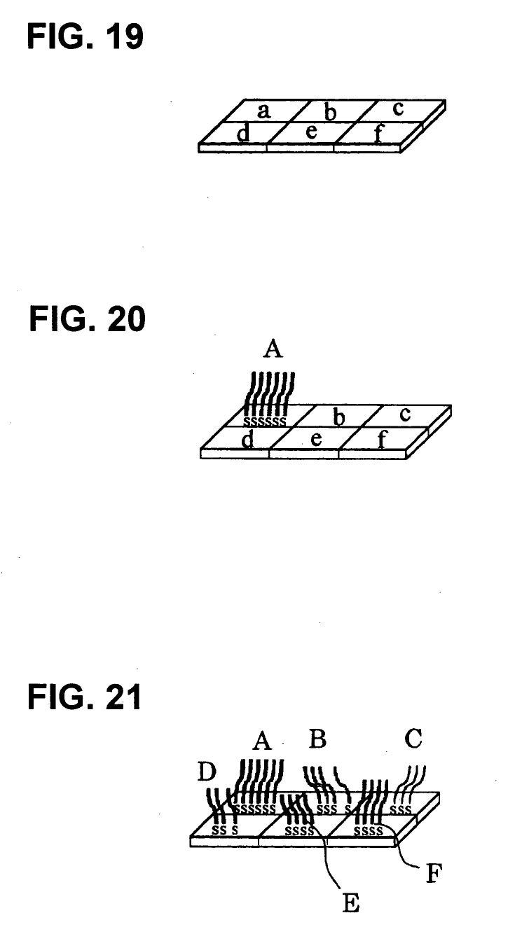

FIGS. 19 to 21 are enlarged model views of the sensor array part 222 of the biochip that are a collection of plural carrier bodies. FIG. 19 illustrates a state before installing analytes. FIG. 20 illustrates a state of analytes A being installed on carrier body a. FIG. 21 illustrates a state after analytes A to F have been installed on carrier bodies a to f.

Analytes are installed on each of carrier bodies a to f on the sensor array part 222 in FIG. 19. The installation sites a-f are wired so that each of them is given an electric potential from the electrode, electrically distinct from each other. When the analytes A to F are installed on the carrier bodies a to f in this order, the procedure is performed as follows:

(1) After completion of the biochip, an analyte solution is poured from the sample inlet 221 of FIGS. 22 and 23. Voltage is applied between the analyte solution and each of the carrier bodies, with the potentials of b to f on the minus side of the potential of a. In this state, an analyte solution comprising analytes A is introduced into the sensor array part. Through this operation, A are attached to and installed on the carrier body a as shown in FIG. 19.

(2) Then, voltage is applied between the analyte solution and each of the carrier bodies, with the potentials of a and c to f on the minus side of the potential of b. In this state, an analyte solution comprising analytes B is introduced into the sensor array part. Through this operation, B are attached to and installed on b. During the course, some of A installed on the carrier body a may flow away. However, it is easy to retain a required amount of A on the carrier body a.

(3) In the same way, analytes are disposed onto each of carrier bodies c to f. In this way, analytes A-F are attached to and installed on the carrier bodies a to f as shown in FIG. 21.

(4) Optimum values of applied voltage and electric potential for the carrier bodies can be determined appropriately by experiments, etc.

FIGS. 20 and 21 are model views illustrating states of analytes being fixed on the analyte installation site, utilizing the following formula:
RSH+Au→AuSR+H⁺+e⁻
One example for the above-described (1) is a case that voltage is applied between the analyte solution and the analyte installation sites so that carrier body a is given a positive potential, while carrier bodies b to f are given a negative potential.

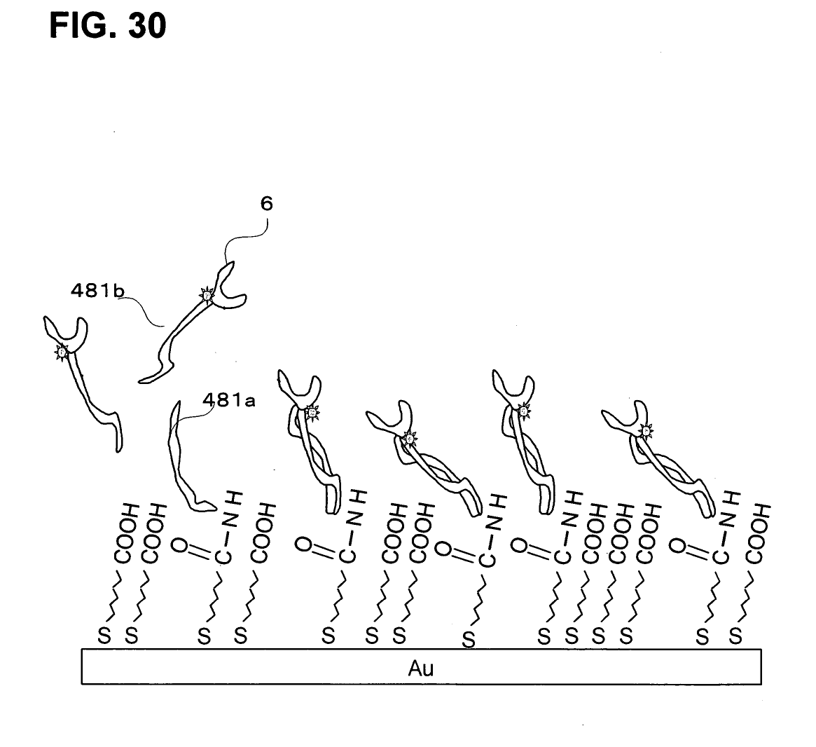

FIGS. 29 and 30 illustrate an example in which DNA analytes are bound, using a Au electrode (carrier body) modified with a SAM formed according the above-described method A by using molecules having a thiol group (—SH) and a carboxy group on both strand ends, respectively.

Through this process, a single-stranded DNA 481a is bound to a carrier body. And a DNA 481b is complementarily bound with the single-stranded DNA 481a to form an analyte according to the present invention. Binding of adenine A with thymine T, and binding of cytosine C with guanine G are examples of such complementary binding. FIG. 30 illustrates a state of DNAs 481b that are floating being selectively attached to DNAs 481a.

In this way, using an antibody (evaluation object binding part) 6 chemically bound with a DNA 481b, it is possible, for example, to bind a protein by an antigen-antibody reaction so as to fix it on a substrate (detection region).

Biochips were prepared in this way. As a result, disuse of manual works for installing analytes was made possible. Shortened manufacturing time, simplified processes, and easier handling of materials to be used, were realized. In addition, analytes can be installed on microscopic areas as well as areas having complex shapes that are difficult to physically access. It is also possible to increase the number of carrier bodies that can be installed per a specific area, or to realize an analyte evaluating device that is smaller than the conventional devices.

In the above, explanation on the fluorescence-labeled part 4 introduced onto the analyte is omitted. It goes without saying that the emission and extinction of the fluorescence-labeled part in this case can be handled in the same way as explained previously.

Also in this example, explanation is made on a case in which a Au electrode is treated as a carrier body, and various different analytes are bound thereon. However, when a SAM as shown in FIG. 29 is formed on a Au electrode to form a carrier body, it is also possible to form carrier bodies having various different SAMs, by using the same procedure. Accordingly, miniaturized analyte evaluating devices having a plurality of different carrier body installation sites are also easily realized, by utilizing these embodiments.

1. An analyte evaluating device comprising:
a carrier body that can be bound with an analyte having a fluorescence-labeled part that can emit fluorescence by light received when the distance between the fluorescence-labeled part and the carrier body is enlarged, the distance between the fluorescence-labeled part and the carrier body being variable by an external action;
a light irradiation device for the fluorescence-labeled part to emit light; and
a fluorescence detecting device for detecting the fluorescence emitted by the fluorescence-labeled part,
wherein the distance between said fluorescence-labeled part and said carrier body can be varied by a responding part equipped on at least one of the analyte and the carrier body.

2. An analyte evaluating device according to claim 1 wherein said external action is an electromagnetic or chemical action.

3. An analyte evaluating device according to claim 2 wherein said carrier body is an electrode and said electromagnetic action is realized by applying an electric potential difference between said electrode and a counter electrode.

4. An analyte evaluating device according to claim 1 wherein said carrier body can be chemically bound with the analyte.

5. An analyte evaluating device according to claim 1 wherein said carrier body has a Au layer on the surface.

6. An analyte evaluating device according to claim 1 wherein said carrier body has an analyte binding part having at least one type of group selected from the class consisting of a carboxy group, thiol group, amino group, thioisocyanate group, isocyanate group and α-keto halide group.

7. An analyte evaluating device according to claim 6 wherein said analyte binding part is bound with the Au layer via a thiol group.

8. An analyte evaluating device according to claim 1 wherein said carrier body can be bound with the analyte by one of the following reactions:
A. a reaction between a carboxy group and an amino group via an imidazole-bound intermediate that is activated by 1-(3-dimethylamino-propyl)-3-ethyl-carbodiimide hydrochloride;
B. a reaction between a carboxy group and an amino group via an N-hydroxysuccinimide-bound or an N-hydroxysuccinimide sulfonic acid-bound intermediate that is activated by 1-(3-dimethylamino-propyl)-3-ethyl-carbodiimide hydrochloride;
C. a reaction between a thiol group and a maleimide group;
D. a reaction between an isocyanate group and an amino group; and
E. a reaction between an α-keto halide group, and an amino group or a thiol group.

9. An analyte evaluating device according to claim 1 wherein said analyte has an evaluation object binding part that has a property to specifically bind to at least one evaluation object selected from the group consisting of proteins, DNAs, RNAs, antibodies, natural or artificial single-stranded nucleotides, natural or artificial double-stranded nucleotides, aptamers, products obtained by limited decomposition of antibodies with a protease, organic compounds having affinity to proteins, biomacromolecules having affinity to proteins, complex materials thereof, and arbitrary combinations thereof.

10. An analyte evaluating device according to claim 9 wherein said evaluation object is a protein.

11. An analyte evaluating device according to claim 1 wherein said responding part can be charged positively or negatively.

12. An analyte evaluating device according to claim 1 wherein said responding part comprises at least one material selected from the group consisting of proteins, DNAs, RNAs, antibodies, natural or artificial single-stranded nucleotides, natural or artificial double-stranded nucleotides, aptamers, products obtained by limited decomposition of antibodies with a protease, organic compounds having affinity to proteins, biomacromolecules having affinity to proteins, complex materials thereof, and arbitrary combinations thereof.

13. An analyte evaluating device according to claim 12 wherein said responding part comprises a natural or artificial single-stranded nucleotide, or a natural or artificial double-stranded nucleotide.

14. An analyte evaluating device according to claim 12 wherein said responding part comprises a Fab fragment or (Fab)₂ fragment of an antibody.

15. An analyte evaluating device according to claim 12 wherein said responding part comprises a fragment derived from an IgG antibody, or a fragment derived from a Fab fragment or (Fab)₂ fragment of an IgG antibody.

16. An analyte evaluating device according to claim 12 wherein said responding part comprises a nucleotide aptamer.

17. An analyte evaluating device according to claim 1 wherein said light irradiation device uses one or more optical fibers.

18. An analyte evaluating device according to claim 1 wherein said light irradiation device is a laser light irradiation device.

19. An analyte evaluating device according to claim 1 wherein evanescent waves can excite the fluorescence-labeled part.

20. An analyte evaluating device according to claim 1 wherein a lens is installed between said light irradiation device and said carrier body.

21. An analyte evaluating device according to claim 20 wherein said lens is a confocal lens.

22. An analyte evaluating device according to claim 1 wherein light can be irradiated from a direction in parallel with the surface of said carrier body.

23. An analyte evaluating device according to claim 1 wherein said carrier body is bound with said analyte.

24. An analyte evaluating device according to claim 1 wherein a plurality of the same type or different types of carrier bodies are installed.

25. An analyte evaluating device according to claim 1 wherein a plurality of the same type or different types of analytes are installed.

26. An analyte evaluating device according to claim 1 wherein a plurality of carrier bodies are installed, and an electric potential is applied to each one of carrier bodies that is different from those for the other carrier bodies so that each carrier body can be bound with a different type of analyte.

27. An analyte evaluating device according to claim 1 wherein an electric potential is applied to each one of plural carrier body installation sites that is different from those for the other carrier body installation sites so that a different type of carrier body is formed on each installation site.

28. An analyte evaluating device having a flow path, an evaluation object capturing part for capturing an evaluation object with a first capture body, and a capture body capturing part for capturing a first capture body that has not captured an evaluation object with a second capture body, installed in this order.

29. An analyte evaluating device according to claim 28 wherein an analyte is evaluated by measuring at least one of the radiation amount of the analyte and the fluorescence intensity at the emission or extinction of fluorescence.

30. An analyte evaluating device according to claim 28 wherein a main body of said analyte evaluating device having an evaluation object capturing part and a capture body capturing part installed in this order, comprises:
a carrier body that can be bound with an analyte having a fluorescence-labeled part that can emit fluorescence by light received when the distance between the fluorescence-labeled part and the carrier body is enlarged, the distance between the fluorescence-labeled part and the carrier body being variable by an external action;
a light irradiation device for the fluorescence-labeled part to emit light; and
a fluorescence detecting device for detecting the fluorescence emitted bythe fluorescence-labeled part.

31. An analyte evaluating device according to claim 1, comprising a flow path, an evaluation object capturing part for capturing an evaluation object with a first capture body, and a capture body capturing part for capturing a first capture body that has not captured an evaluation object with a second capture body, installed in this order.

32. An analyte evaluating device according to claim 28, wherein said evaluation object capturing part comprises a carrier body that can be bound with and detached from the first capture body by the presence or absence of an external action.

33. An analyte evaluating device according to claim 28, wherein said capture body capturing part comprises a carrier body that can be bound with and detached from the second capture body by the presence or absence of an external action.

34. An analyte evaluating device according to claim 32 or 33, wherein the presence or absence of said external action is the presence or absence of an electromagnetic or chemical action.

35. An analyte evaluating device according to claim 34, wherein the presence or absence of said electromagnetic action is created by applying or not applying an electric potential difference between an electrode and a counter electrode.

36. An analyte evaluating device according to claim 28, wherein said first capture body has a property to be specifically bound with the evaluation object.

37. An analyte evaluating device according to claim 28, wherein said first capture body can be specifically bound with the evaluation object and the second capture body at the same site.

38. An analyte evaluating device according to claim 28, wherein at least one of said first and second capture bodies is bound to a Au layer via a thiol group.

39. An analyte evaluating device according to claim 28, wherein said first capture body has a property to be specifically bound with at least one evaluation object selected from the group consisting of proteins, DNAs, RNAs, antibodies, natural or artificial single-stranded nucleotides, natural or artificial double-stranded nucleotides, aptamers, products obtained by limited decomposition of antibodies with a protease, organic compounds having affinity to proteins, biomacromolecules having affinity to proteins, complex materials thereof, and arbitrary combinations thereof.

40. An analyte evaluating device according to claim 39, wherein said evaluation object is a protein.

41. An analyte evaluating device according to claim 28, wherein at least one of said first and second capture bodies can be charged positively or negatively.

42. An analyte evaluating device according to claim 28, wherein at least one of said first and second capture bodies comprises at least one material selected from the group consisting of proteins, DNAs, RNAs, antibodies, natural or artificial single-stranded nucleotides, natural or artificial double-stranded nucleotides, aptamers, products obtained by limited decomposition of antibodies with a protease, organic compounds having affinity to proteins, biopolymers having affinity to proteins, complex materials thereof, and arbitrary combinations thereof.

43. An analyte evaluating device according to claim 42, wherein at least one of said first and second capture bodies comprises a natural or artificial single-stranded nucleotide, or a natural or artificial double-stranded nucleotide.

44. An analyte evaluating device according to claim 42, wherein at least one of said first and second capture bodies comprises a Fab fragment or (Fab)₂ fragment of an antibody.

45. An analyte evaluating device according to claim 42, wherein at least one of said first and second capture bodies comprises a fragment derived from an IgG antibody, or a fragment derived from a Fab fragment or (Fab)₂ fragment of an IgG antibody.

46. An analyte evaluating device according to claim 42, wherein at least one of said first and second capture bodies comprises a nucleotide aptamer.

47. An analyte evaluating device according to claim 28, wherein at least one of said first and second capture bodies comprises at least one type of group selected from the class consisting of a carboxy group, thiol group, amino group, thioisocyanate group, isocyanate group and α-keto halide group.

48. An analyte evaluating device according to claim 28, wherein said first capture body can be bound with the evaluation object, or said second capture body can be bound with the first capture body, or both of the binding is possible, by one of the below-described reactions A to E.
A. A reaction between a carboxy group and an amino group via an imidazole-bound intermediate that is activated by 1-(3-dimethylamino-propyl)-3-ethyl-carbodiimide hydrochloride,
B. a reaction between a carboxy group and an amino group via an N-hydroxysuccinimide-bound or an N-hydroxysuccinimide sulfonic acid-bound intermediate that is activated by 1-(3-dimethylamino-propyl)-3-ethyl-carbodiimide hydrochloride,
C. a reaction between a thiol group and a maleimide group,
D. a reaction between an isocyanate group and an amino group, and
E. a reaction between an α-keto halide group, and an amino group or thiol group.

49. An analyte evaluating device according to claim 28, wherein said carrier body of the evaluation object capturing part has a Au layer on the surface, and said first capture body can be bound to and detached from the Au layer via a thiol group.

50. An analyte evaluating device according to claim 28, wherein said carrier body of the capture body capturing part has a Au layer on the surface, and the second capture body can be bound to and detached from the Au layer via a thiol group.

51. An analyte evaluating device according to claim 28, wherein at least one of the outlets of said evaluation object capturing part and said capture body capturing part has a bottle-neck part to prevent said first or second capture body from exiting from the outlet.

52. A method for evaluating an analyte comprising:
using an analyte evaluating device according to claim 1 or 28;
binding the analyte with the carrier body;
changing the distance between the fluorescence-labeled part and the carrier body by an external action;
irradiating light from the light irradiation device; and
detecting fluorescence emitted from the fluorescence-labeled part with the fluorescence detecting device.

53. A method for evaluating an analyte according to claim 52, wherein light is irradiated from a direction in parallel with the surface of said carrier body.

54. A method for evaluating an analyte according to claim 52, wherein said analyte is bound with an evaluation object before the analyte is bound with the carrier body.

55. A method for evaluating an analyte according to claim 52, wherein each carrier body is given an electric potential different from those of the other carrier bodies so that a different type of analyte is disposed on each carrier body.

56. A method for evaluating an analyte according to claim 52, wherein an electrode is used as the carrier body, and the electromagnetic action is realized by providing a potential difference having either one of a constant value, a pulse value, a value changing in a stepwise manner, and a periodically changing value or a combination thereof, between the electrode and an counter electrode.

57. A method for evaluating an analyte according to claim 52, wherein at least one physical property selected from the group consisting of generation or non-generation of fluorescence emission, the rate of increase in the fluorescence intensity, the rate of decrease in the fluorescence intensity, the peak fluorescence intensity and the rate of change of the peak fluorescence intensity, is measured.

58. A method for manufacturing an analyte evaluating device according to claim 1 or 28, wherein the carrier body of the analyte evaluating device or a main body thereof is prepared by treating a Au layer in an aqueous solution by either one of the following reactions A to E.
A. A reaction between a carboxy group and an amino group via an imidazole-bound intermediate that is activated by 1-(3-dimethylamino-propyl)-3-ethyl-carbodiimide hydrochloride,
B. a reaction between a carboxy group and an amino group via an N-hydroxysuccinimide-bound or an N-hydroxysuccinimide sulfonic acid-bound intermediate that is activated by 1-(3-dimethylamino-Kaoru propyl)-3-ethyl-carbodiimide hydrochloride,
C. a reaction between a thiol group and a maleimide group,
D. a reaction between an isocyanate group and an amino group, and
E. a reaction between an α-keto halide group, and an amino group or thiol group.

59. A method for manufacturing an analyte evaluating device according to claim 28, wherein the Au layer of at least one of said first capture body and second capture body is treated in an aqueous solution according to either one of the following reactions A to E.
A. A reaction between a carboxy group and an amino group via an imidazole-bound intermediate that is activated by 1-(3-dimethylamino-propyl)-3-ethyl-carbodiimide hydrochloride,
B. a reaction between a carboxy group and an amino group via an N-hydroxysuccinimide-bound or an N-hydroxysuccinimide sulfonic acid-bound intermediate that is activated by 1-(3-dimethylamino-propyl)-3-ethyl-carbodiimide hydrochloride,
C. a reaction between a thiol group and a maleimide group,
D. a reaction between an isocyanate group and an amino group, and
E. a reaction between an α-keto halide group, and an amino group or thiol group.

60. A method for manufacturing an analyte evaluating device according to claim 1 or 28, wherein an electric potential is applied to each one of plural carrier bodies that is different from those for the other carrier bodies so that each carrier body is bound with a different type of analyte.

61. A method for manufacturing an analyte evaluating device according to claim 1 or 28, wherein an electric potential is applied to each one of plural carrier body installation sites that is different from those of the other carrier body installation sites so that each carrier body installation site is bound with a different type of carrier body.

62. A method for manufacturing an analyte evaluating device according to claim 1 or 28, wherein a different type of analyte is given to each carrier body after a cover is installed onto the analyte evaluating device.

63. A method for manufacturing an analyte evaluating device according to claim 1 or 28, wherein a different type of carrier body is given to each carrier body installation site after a cover is installed onto the analyte evaluating device.

64. A method for evaluating an analyte according to claim 53, wherein said analyte is bound with an evaluation object before the analyte is bound with the carrier body.

65. A method for evaluating an analyte according to claim 53, wherein each carrier body is given an electric potential different from those of the other carrier bodies so that a different type of analyte is disposed on each carrier body.

66. A method for evaluating an analyte according to claim 53, wherein an electrode is used as the carrier body, and the electromagnetic action is realized by providing a potential difference having either one of a constant value, a pulse value, a value changing in a stepwise manner, and a periodically changing value or a combination thereof, between the electrode and an counter electrode.

67. A method for evaluating an analyte according to claim 53, wherein at least one physical property selected from the group consisting of generation or non-generation of fluorescence emission, the rate of increase in the fluorescence intensity, the rate of decrease in the fluorescence intensity, the peak fluorescence intensity and the rate of change of the peak fluorescence intensity, is measured.
